(19) United States
(12) Patent Application Publication
(10) Pub. No.: US 2022/0175953 A1
(43) Pub. Date: Jun. 9, 2022

(54) BIOMIMETIC ANISOTROPIC POLYMERIC PARTICLES WITH NATURALLY DERIVED CELL MEMBRANES FOR ENHANCED DRUG DELIVERY

(71) Applicant: The Johns Hopkins University, Baltimore, MD (US)

(72) Inventors: Jordan J. Green, Nottingham, MD (US); Randall A. Meyer, Baltimore, MD (US); Elana Ben-Akiva, Baltimore, MD (US)

(21) Appl. No.: 17/550,542

(22) Filed: Dec. 14, 2021

Related U.S. Application Data
(63) Continuation of application No. 16/089,787, filed on Sep. 28, 2018, now abandoned.
(60) Provisional application No. 62/314,127, filed on Mar. 28, 2016.

(51) Int. Cl. A61K 47/69; A61K 9/50; A61K 39/395; A61K 49/00

(52) U.S. Cl. CPC A61K 47/6901; A61K 49/0002; A61K 39/3955; A61K 9/5068

(57) ABSTRACT

The presently disclosed subject matter provides a biomimetic particle platform that can be used to simulate natural cells found throughout the body. The particle comprises a polymeric core of defined shape, size, and mechanical properties and a surface comprising naturally derived cell membranes, such as red blood cells or platelets. Together these features enable a level of biomimicry that can be appropriated for various drug delivery applications and cell engineering applications.

### CROSS-REFERENCE TO RELATED APPLICATIONS

This application is a continuation of U.S. patent application Ser. No. 16/089,787, filed Sep. 28, 2018, which is a § 371 National Entry of PCT/US2017/024542, filed Mar. 28, 2017, which claims priority to U.S. Provisional Application No. 62/314,127 filed on Mar. 28, 2016, each of which is incorporated fully herein by reference.

### FEDERALLY SPONSORED RESEARCH OR DEVELOPMENT

This invention was made with government support under EB016721 awarded by the National Institutes of Health. The government has certain rights in the invention.

### BACKGROUND

Although biomimetic polymeric particles have been used for drug delivery, these particles have not been able to mimic the natural cell in terms of size, shape, mechanical properties, and surface features.

### SUMMARY

In one aspect, disclosed are biomimetic particles selected from the group consisting of: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c).

In certain aspects, disclosed are kits comprising a biomimetic particle or composition selected from the group consisting of: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c).

In some aspects, disclosed are methods for delivering a drug to a diseased cell and/or tissue of a patient, the method comprising: (a) administering to a patient a biomimetic particle loaded with an agent for treating a disease or disorder, wherein the biomimetic particle is selected from the group consisting of: (i) a first biomimetic particle comprising: (1) a three-dimensional microparticle or nanoparticle; and (2) a naturally derived cell membrane, wherein the naturally derived cell membrane is not derived from a red blood cell; (ii) a second biomimetic particle comprising: (1) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (2) a naturally derived cell membrane; (iii) a third biomimetic particle comprising: (1) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (2) a naturally derived cell membrane; or (iv) a composition comprising (i), (ii), or (iii), or combinations of (i), (ii) and (iii); and (b) allowing the biomimetic particle to target a diseased cell and/or tissue of the patient, thereby delivering the drug to the diseased cell and/or tissue of the patient.

In another aspect, disclosed are methods for treating a disease or disorder in a patient in need thereof, the method comprising: (a) administering to a patient an effective amount of a biomimetic particle loaded with an agent for treating a disease or disorder, wherein the biomimetic particle is selected from the group consisting of: (i) a first biomimetic particle comprising: (1) a three-dimensional microparticle or nanoparticle; and (2) a naturally derived cell membrane, wherein the naturally derived cell membrane is not derived from a red blood cell; (ii) a second biomimetic particle comprising: (1) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (2) a naturally derived cell membrane; (iii) a third biomimetic particle comprising: (1) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (2) a naturally derived cell membrane; or (iv) a composition comprising (i), (ii), or (iii), or combinations of (i), (ii) and (iii); and (b) allowing the biomimetic particle to target a diseased cell and/or tissue of the patient, thereby treating the disease or disorder in the patient.

In other aspects, disclosed are methods for treating a disease or disorder in a patient in need thereof, the method comprising administering to a patient an effective amount of a biomimetic particle for treating a disease or disorder, wherein the biomimetic particle is selected from the group consisting of: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c); thereby treating the disease or disorder in the patient.

In certain aspects, disclosed are methods for imaging a diseased cell and/or tissue of a patient, the method comprising: (a) administering to a patient a biomimetic particle comprising: (i) a three-dimensional microparticle or nanoparticle and a naturally derived cell membrane, wherein the naturally derived cell membrane is not derived from a red blood cell; or (ii) a three-dimensional microparticle or nanoparticle having an asymmetrical shape, wherein the asymmetrical shape has at least one surface having a radius of curvature along at least one axis selected from one of the following ranges: (1) about 1 nm to about 10 nm; (2) about 11 nm to about 100 nm; (3) about 101 nm to about 400 nm; (4) about 401 nm to about 1 μm; (5) about 10 μm to about 20 μm; (6) about 20 μm to about 100 μm; and (7) about 101 μm to about 1 mm; and a naturally derived cell membrane; (iii) an imaging agent; and (iv) a targeting agent; wherein upon administration, the biomimetic particle targets a diseased cell and/or tissue of the patient; and (b) imaging the diseased cell and/or tissue of the patient.

In other aspects, disclosed are methods for administering a biotinylated drug to a patient, the method comprising administering to a patient a biomimetic particle conjugated with a biotin-binding protein or fragment thereof, wherein the biomimetic particle is selected from the group consisting of: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c).

In some aspects, the biomimetic particle comprising the three-dimensional microparticle or nanoparticle having an asymmetrical shape comprises a naturally derived cell membrane derived from a red blood cell. In some aspects, the naturally derived cell membrane is a cell membrane derived from a platelet or a stem cell.

In some aspects, the surface of the biomimetic particle is functionalized with a targeting agent, a therapeutic agent, and/or an imaging agent. In some aspects, the targeting agent, therapeutic agent, and/or imaging agent is selected from the group consisting of a small molecule, carbohydrate, sugar, protein, peptide, nucleic acid, antibody or antibody fragment thereof, hormone, hormone receptor, receptor ligand, and cancer cell specific ligand.

In some aspects, imaging the diseased cell and/or tissue of the patient occurs by bioluminescent imaging, fluorescent imaging, x-ray computed tomography (CT), magnetic resonance imaging (MRI), positron emission tomography (PET), single-photon emission computed tomography (SPECT), and/or X-ray, and operable combinations thereof.

In some aspects, the biomimetic particle is biodegradable and/or biocompatible in the patient. In some aspects, the microparticle or nanoparticle comprises a polymeric matrix. In some aspects, the polymeric matrix is selected from the group consisting of poly(D,L-lactide-co-glycolide) (PLGA), poly (D,L-lactic acid) (PDLLA), polycaprolactone (PCL), polyglycolic acid (PGA), polylactic acid (PLA), poly(acrylic acid) (PAA), poly-3-hydroxybutyrate (P3HB) and poly(hydroxybutyrate-co-hydroxyvalerate). In some aspects, the biomimetic particle further comprises a biodegradable polymer or blends of polymers blended with a nondegradable polymer. In some aspects, the biomimetic particle ranges in size from about 10 nanometers to about 500 microns.

In some aspects, the polymeric matrix is stretched from above the polymer transition temperature up to the polymer degradation temperature to form an anisotropic polymeric matrix. In some aspects, the polymeric matrix is stretched at a temperature above but close to the polymer transition temperature to form an anisotropic polymeric matrix. In some aspects, stretching of the polymeric matrix causes the polymeric matrix to change from a generally spherical shape to an anisotropic shape.

In some aspects, the biotin-binding protein or fragment thereof is selected from the group consisting of avidin, streptavidin, neutravidin, and a fragment thereof. In some aspects, the biotinylated drug is a biotinylated antibody. In some aspects, the biotinylated antibody is specific for CD28.

In some aspects, the disease or disorder is selected from the group consisting of excessive bleeding, thalassemia, thrombopenia, thrombasthenia, cancer, an infectious disease, and a degenerative disease.

In some aspects, disclosed are uses of a composition comprising a biomimetic particle selected from the group consisting of: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c) for the treatment of a disease or disorder.

In certain aspects, disclosed are uses of a composition comprising a biomimetic particle selected from the group consisting of: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (f) about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c) for the manufacture of a medicament for the treatment of a disease or disorder.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

FIG. 1A, FIG. 1B, FIG. 1C, FIG. 1D, FIG. 1E, and FIG. 1F show that different biomaterials can be made into ellipsoidal microparticles utilizing the automated thin film stretching procedure. In addition to PLGA, PCL (FIG. 1A and FIG. 1D) and a PLGA/PBAE blend (FIG. 1B and FIG. 1E) can be fabricated into ellipsoidal shapes by the disclosed automated stretching method. FIG. 1C shows a particle size distribution plot demonstrating that PCL microparticles have a larger size than PLGA/PBAE microparticles (x axis units are microns). FIG. 1F shows the aspect ratio analysis of each sample demonstrating that despite size differences, similar aspect ratios can be attained for each particle.

FIG. 2A, FIG. 2B, FIG. 2C, FIG. 2D, FIG. 2E, and FIG. 2F show that non-spherical particles of various shapes can be synthesized with the disclosed two-dimensional automated thin film stretching method. Spherical microparticles (FIG. 2A) can be stretched 1.25× by 1.25× (FIG. 2B), 1.25× by 1.5× (FIG. 2C), 1.5× by 1.5× (FIG. 2D), and 1.75× by 1.75× (FIG. 2E) to create particles of various flattened oblate ellipsoidal shapes. FIG. 2F shows that aspect ratio analysis of 1.5× by 1.5× stretched particles reveals that the aspect ratio of one is roughly maintained through the 2D stretching procedure.

FIG. 3A, FIG. 3B, FIG. 3C, and FIG. 3D show that the shape of the particle depends on the temperature at which it was stretched. Spherical microparticles were stretched at 70° C. (FIG. 3A), 80° C. (FIG. 3B), 90° C. (FIG. 3C), and 100° C. (FIG. 3D) in 2 dimensions at a fold of 1.5× by 1.5×. As the temperature increases, the frequency of dimpled particles increases.

FIG. 4A and FIG. 4B show that non spherical particles can be coated with a biomimetic red blood cell membrane. Spherical (FIG. 4A) and prolate ellipsoidal (FIG. 4B) particles were coated with red blood cell membranes. (Left) particles only in blue, (middle) RBC membranes only in red, (right) merge particle and membrane channels.

FIG. 5A and FIG. 5B show characteristic avidin content. FIG. 5A shows total protein conjugation amount of fluorescent avidin to spherical and ellipsoidal supported lipid bilayers. FIG. 5B shows efficiency of conjugation for various ratios of avidin to mass of particles in synthesis. A two-way ANOVA was performed to analyze statistical differences in the efficiency data set: p=0.0303 for shape/dose interaction, p=0.0057 for shape impact on results, and p=0.0013 for dose impact on results. There was no significant difference between shapes at any dose tested as evaluated by Bonferroni's post test (p>0.05) except for at 1 μg avidin/mg PLGA (p<0.01).

FIG. 6A, FIG. 6B, FIG. 6C and FIG. 6D show interchangeable protein surface conjugation to spherical and ellipsoidal lipid bilayers. Spherical (FIG. 6A) and ellipsoidal (FIG. 6B) supported lipid bilayers encapsulating 7-AMC (blue) conjugated to avidin-biotin-fluorophore (magenta) conjugate on the surface. FIG. 6C shows that total protein captured by particles exhibits dependency on the amount of protein dosed in synthesis. FIG. 6D shows that efficiency of conjugation between spherical and non-spherical supported lipid bilayers at various doses is similar. Error bars represent SEM of 3 replicates.

FIG. 7A, FIG. 7B and FIG. 7C show red blood cell ghosts can be fused with synthetic lipids. (FIG. 7A) Brightfield, (FIG. 7B) fluorescent, and (FIG. 7C) merged images of red blood cell ghosts fused with a lipid vesicles containing a PEGylated and non-PEGylated fluorophore lipid. The colocalization of the fluorescence with the ghosts on brightfield confirms integration of the synthetic lipids in the ghosts.

FIG. 8A and FIG. 8B show functionalized red blood cell ghosts can be coated onto PLGA particles. Particles were (FIG. 8A) coated with fluorescent membranes derived from red blood cells or (FIG. 8B) mock sonicated without red blood cell membranes. Left panels show particles in blue, middle panels show red blood cell membranes in red, and the right panels shows the merged images. The coating of the blue polymeric particles with the red naturally derived cell membranes demonstrates the feasibility and flexibility of this approach.

FIG. 9 shows biodegradable polymeric particles (blue) coated by RBC membranes (red). In the presence of the RBC membrane, there is a region of red that outlines the particle indicating successful coating with the red blood cell membrane.

FIG. 10 shows spherical particles coated by red blood cell membrane derived material. Particles (blue, left) were coated with RBC membranes (red, middle) and the merged image (right) fluorescence profile (bottom) was analyzed. As shown when the fluorescence is tracked on a line through the particle, the red signal is enriched on the edges and the particle signal is enriched in the center indicating successful coating.

FIG. 11 shows prolate ellipsoidal particles coated by red blood cell membrane derived material. Particles (blue, left) were coated with RBC membranes (red, middle) and the merged image (right) fluorescence profile (bottom) was analyzed. As shown when the fluorescence is tracked on a line through the particle, the red signal is enriched on the edges and the particle signal is enriched in the center indicating successful coating.

FIG. 12 shows oblate ellipsoidal particles coated by red blood cell membrane derived material. Particles (blue, left) were coated with RBC membranes (red, middle) and the merged image (right) fluorescence profile (bottom) was analyzed. As shown when the fluorescence is tracked on a line through the particle, the red signal is enriched on the edges and the particle signal is enriched in the center indicating successful coating.

FIG. 13 shows red blood cell membranes on the surface of particles display cell membrane like lateral fluidity. Spherical and RBC shaped particles exhibit fluorescence recovery evident of lateral mobility of the lipids in the cell membranes.

FIG. 14 shows red blood cell membranes on the surface of 1-D stretched particles display cell membrane like lateral fluidity. The diffusion coefficients were extracted using a Gaussian 2D diffusion model and determined to be on the order of lipids found in natural cell membranes.

FIG. 15 shows RBC membranes maintain biologically active surface proteins during the coating process. Fluorescently labeled anti-CD47 was incubated with the particles for 1 hour and the particles were washed and analyzed by fluorescence. The particles coated with the red blood cell membranes had significantly higher fluorescence compared to the control conditions.

FIG. 16 shows spherical particles coated with platelet derived material. Particles (blue, left) were coated with platelet membranes (yellow, middle) and the merged image (right) fluorescence profile (bottom) was analyzed. As shown when the fluorescence is tracked on a line through the particle, the yellow signal is enriched on the edges and the particle signal is enriched in the center indicating successful coating.

FIG. 17 shows prolate ellipsoidal particles coated with platelet derived material. Particles (blue, left) were coated with platelet membranes (yellow, middle) and the merged image (right) fluorescence profile (bottom) was analyzed. As shown when the fluorescence is tracked on a line through the particle, the yellow signal is enriched on the edges and the particle signal is enriched in the center indicating successful coating.

FIG. 18 shows oblate ellipsoidal particles coated with platelet derived material. Particles (blue, left) were coated with platelet membranes (yellow, middle) and the merged image (right) fluorescence profile (bottom) was analyzed. As shown when the fluorescence is tracked on a line through the particle, the yellow signal is enriched on the edges and the particle signal is enriched in the center indicating successful coating.

FIG. 19 shows spherical particles coated with activated platelet derived material. Particles (blue, left) were coated with activated platelet membranes (yellow, middle) and the merged image (right) fluorescence profile (bottom) was analyzed. As shown when the fluorescence is tracked on a line through the particle, the yellow signal is enriched on the edges and the particle signal is enriched in the center indicating successful coating.

FIG. 20 shows prolate ellipsoidal particles coated with activated platelet derived material. Particles (blue, left) were coated with activated platelet membranes (yellow, middle) and the merged image (right) fluorescence profile (bottom) was analyzed. As shown when the fluorescence is tracked on a line through the particle, the yellow signal is enriched on the edges and the particle signal is enriched in the center indicating successful coating.

FIG. 21 shows oblate ellipsoidal particles coated with activated platelet derived material. Particles (blue, left) were coated with activated platelet membranes (yellow, middle) and the merged image (right) fluorescence profile (bottom) was analyzed. As shown when the fluorescence is tracked on a line through the particle, the yellow signal is enriched on the edges and the particle signal is enriched in the center indicating successful coating.

FIG. 22 shows biodegradable particles coated with cell membranes derived from dendritic cells. Particles (green, left) were coated with labeled dendritic cell membranes (red, middle), and merged on the right to highlight the membrane coating.

FIG. 23 shows biodegradable particles coated with a cell membrane via a co-extrusion process. As shown, the membrane coating is not uniform using a co-extrusion process.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: www.ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

In some embodiments, the presently disclosed subject matter provides a biomimetic particle platform that can be used to simulate natural cells found throughout the body. The biomimetic particle comprises a polymeric construct and a naturally derived cell membrane. In some embodiments, the presently disclosed biomimetic particle mimics the natural cell in terms of size, shape, mechanical properties, and surface features. In some embodiments, the presently disclosed subject matter allows multiple levels of cellular biomimicry including simulation of the anisotropic shape of cells through thin film stretching, reproduction of cellular mechanical properties through material selection, control over particle size, and precise surface mimicry enabled by cellular membrane derived vesicle fusion. Together these features enable a level of biomimicry that can be appropriated for various drug delivery applications and cell engineering applications.

I. Biomimetic Particles

In some embodiments, the presently disclosed subject matter provides a biomimetic particle selected from the group consisting of: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c).

As used herein, by “biomimetic particle,” it is meant a particle that has properties that mimic a natural cell. For example, the presently disclosed biomimetic particles comprise naturally derived cell membranes. In some embodiments, normal cells found in a body, such as platelets or stem cells, are processed into submicron membrane vesicles and then fused with the presently disclosed polymeric particles.

As used herein, the term “nanoparticle,” refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, and 1000 nm and all integers and fractional integers in between). In some embodiments, the nanoparticle has at least one dimension, e.g., a diameter, of about 100 nm. In some embodiments, the nanoparticle has a diameter of about 200 nm. In some embodiments, a 200 nm membrane filter is used for extrusion. In other embodiments, the nanoparticle has a diameter of about 500 nm. In yet other embodiments, the nanoparticle has a diameter of about 1000 nm (1 μm). In such embodiments, the particle also can be referred to as a “microparticle.” Thus, the term “microparticle” includes particles having at least one dimension in the range of about one micrometer (μm), i.e., 1×10⁻⁶ meters, to about 1000 μm. The term “particle” as used herein is meant to include nanoparticles and microparticles. In some embodiments, the biomimetic particle ranges in size from about 10 nanometers to about 500 microns. In some embodiments, the biomimetic particle ranges in size from about 50 nanometers to about 5 microns. In some embodiments, the biomimetic particle is at least 10, 20, 30, 40, or 50 nanometers in size. In some embodiments, the biomimetic particle is less than 500, 400, 300, 200, 100, 50, 10, or 5 microns in size. In some embodiments, the biomimetic particles are small enough to have an enhanced permeability and retention (EPR) effect, such that they tend to accumulate in tumor tissue rather than normal tissue. Size of nanoparticles and microparticles can be measured by scanning electron microscope and/or transmission electron microscope and then analyzed, e.g., particle diameter. In addition to electron microscopy, light scattering methods, such as dynamic light scattering, may be used to analyze particle size (e.g., hydrodynamic diameter).

In some embodiments, the microparticle or nanoparticle has an aspect ratio ranging from about 1 to about 5. In some embodiments, the aspect ratio has a range from about 5 to about 10. In some embodiments, the aspect ratio has a range from about 10 to about 100.

As used herein, the term “naturally derived cell membrane” refers to a cell membrane that is made from a cell found in the body, such as a stem cell, a blood cell (e.g., a platelet, red blood cell, white blood cell), a fat cell, a skin cell, an endothelial cell, a nerve cell, and the like. In some embodiments, the naturally derived cell membrane is not derived from a red blood cell. In some embodiments, the naturally derived cell membrane may be derived from a red blood cell. In some embodiments, the naturally derived cell membrane is a cell membrane derived from a platelet. For example, the cell membrane may be derived from a resting platelet or an activated platelet. In some embodiments, the naturally derived cell membrane is a cell membrane derived from a stem cell. In some embodiments, the naturally derived cell membrane is a cell membrane derived from an immune cell, such as a dendritic cell or a T cell.

The naturally derived cell membrane can be supported on the three-dimensional microparticle or nanoparticle as a lipid bilayer. The naturally derived cell membrane may be supported on the micro/nano particle through non-covalent interactions. In some embodiments, the naturally derived cell membrane can be arranged on the surface of the three-dimensional microparticle or nanoparticle through a sonication method. For example, the naturally derived cell membrane and the three-dimensional microparticle or nanoparticle may be sonicated together to provide a naturally derived cell membrane supported on the three-dimensional microparticle or nanoparticle as a lipid bilayer.

The naturally derived cell membrane may also include additional components that were not naturally derived from a cell membrane. For example, the naturally derived cell membrane may include other added lipid fractions, such as 1,2-dioleoyl-sn-glycero-3-phosphocholine (DOPC) and 1,2-Distearoyl-sn-glycero-3-phosphoethanolamine (DSPE), or other added protein fractions. The naturally derived cell membrane may include from about 80 wt % to about 100 wt % naturally derived cell membrane components, such as from about 85 wt % to about 100 wt %, from about 90 wt % to about 100 wt %, or from about 95 wt % to about 99 wt % naturally derived cell membrane components.

In some embodiments, the naturally derived cell membrane may include from about 0.01 wt % to about 5 wt % added lipid, such as from about 0.01 wt % to about 1 wt %, from about 0.01 wt % to about 0.1 wt %, or from about 0.01 wt % to about 0.05 wt %.

Depending on the application, the added lipid fraction(s) may be functionalized with different agents, as described below. The added lipid fraction(s) may include more than one agent. For example, the naturally derived cell membrane may include an added lipid fraction comprising PEG and another added lipid fraction comprising a fluorophore.

The naturally derived cell membrane may have properties that a cell membrane would have in its native form. For example, the naturally derived cell membranes of the disclosed biomimetic particles may demonstrate lateral fluidity on par with its natural counterparts. In some embodiments, the naturally derived cell membrane may have a diffusion coefficient of from about 1×10¹⁰ cm²/s to about 3×10¹⁰ cm²/s. In addition, the naturally derived cell membranes can maintain the biological activity of their surface proteins, as would be seen in their native counterparts.

In some embodiments, the biomimetic particle comprises a particle that has an asymmetrical shape or is anisotropic. The term “anisotropic” refers to a microparticle or nanoparticle that is non-spherical. In some embodiments, the anisotropic feature of the presently disclosed biomimetic particle makes it more resistant to phagocytosis as compared to a similar spherical biomimetic particle. In some embodiments, the presently disclosed anisotropic biomimetic particles are at least 10% (e.g., 20%, 30%, 40%, or more) more resistant to phagocytosis than similar spherical biomimetic particles. As used herein, the term “phagocytosis” refers to the process by which a cell engulfs a solid particle to form an internal vesicle known as a phagosome. In some embodiments, the biomimetic particle has an asymmetrical shape and a naturally derived cell membrane from a red blood cell. In some embodiments, the biomimetic particle has an asymmetrical shape and a naturally derived cell membrane from a platelet. In some embodiments, the biomimetic particle has an asymmetrical shape and a naturally derived cell membrane from a stem cell.

The biomimetic particles may comprise an agent, e.g., a diagnostic agent (an agent that can be used to diagnose a disease or condition), an imaging agent (an agent that can be used to reveal and/or define the localization of a disease or condition), a targeting agent (an agent that can target a specific kind of cell or tissue, such as a cancer cell), a theranostic agent (an agent that can diagnose and also treat a disease or condition), a therapeutic agent (an agent that can treat a disease or condition), and the like, as well as combinations thereof. In some embodiments, the surface of the biomimetic particle is functionalized with a targeting agent, a therapeutic agent, and/or an imaging agent.

In some embodiments, the naturally derived cell membrane includes the agent(s). For example, the surface of the biomimetic particle may be functionalized with the agent. In some embodiments, the three-dimensional microparticle or nanoparticle may include the agent(s). In still other embodiments, the naturally derived cell membrane and the three-dimensional microparticle or nanoparticle may both include the agent(s).

The naturally derived cell membrane may include the agent at from about 0 wt % to about 100 wt % (by weight of the agent), such as from about 10 wt % to about 90 wt %, from about 25 wt % to about 75 wt %, or from about 25 wt % to about 50 wt %. In addition, the three-dimensional microparticle or nanoparticle may include the agent in weight percentages as described above for the naturally derived cell membrane.

In some embodiments, the agent is a DNA, RNA, polypeptide, antibody, antibody fragment, antigen, carbohydrate, protein, peptide, enzyme, amino acid, hormone, steroid, vitamin, drug, virus, polysaccharide, lipid, lipopolysaccharide, glycoprotein, lipoprotein, nucleoprotein, oligonucleotide, immunoglobulin, albumin, hemoglobin, coagulation factor, peptide hormone, protein hormone, non-peptide hormone, interleukin, interferon, cytokine, peptides comprising a tumor-specific epitope, cell, cell-surface molecule, cell adhesion peptide, cell-binding peptide, cell receptor ligand, small organic molecule, small organometallic molecule, nucleic acid, oligonucleotide, transferrin, metabolites thereof, and antibodies or agents that bind to any of the above substances.

Non-limiting examples of a targeting agent include a sugar, peptide, antibody or antibody fragment, hormone, hormone receptor, receptor ligand, and the like. In some embodiments, the targeting agent is a moiety that has affinity for a tumor associated factor, such as RGD sequences, low-density lipoprotein sequences, a NAALADase inhibitor, epidermal growth factor, and other agents that bind with specificity to a target cell. In some embodiments, the targeting agent is a moiety that has affinity for an inflammatory factor (e.g., a cytokine or a cytokine receptor moiety (e.g., TNF-α receptor)). Antibodies can be generated to allow for the targeting of antigens or immunogens (e.g., tumor, tissue or pathogen specific antigens) on various biological targets (e.g., pathogens, tumor cells, and normal tissue). Such antibodies include, but are not limited to polyclonal, monoclonal, chimeric, single chain, Fab fragments, and a Fab expression library.

In some embodiments, the targeting agent targets a diseased cell and/or tissue of a patient. In some embodiments, the target location within the patient may be any site that the biomimetic particles are capable of being targeted to, particularly a diseased site. Non-limiting examples of target locations include the brain, colon, breasts, prostate, liver, kidneys, lungs, esophagus, head and neck, ovaries, cervix, stomach, colon, rectum, bladder, uterus, testes, and pancreas.

In some embodiments, the target location is a cancer site. A “cancer site” in a patient refers to a site showing the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. Non-limiting examples of cancer that can be treated with the presently disclosed biomimetic particles include brain, colon, breast, prostate, liver, kidney, lung, esophagus, head and neck, ovarian, cervical, stomach, colon, rectal, bladder, uterine, testicular, and pancreatic.

In some embodiments, the presently disclosed biomimetic particles further comprise a therapeutic agent. Non-limiting examples of therapeutic agents include small molecules, such as small organic or inorganic molecules; saccharides; oligosaccharides; polysaccharides; a biological macromolecule selected from the group consisting of peptides, proteins, peptide analogs and derivatives; peptidomimetics; nucleic acids, such as DNA, RNA interference molecules, selected from the group consisting of siRNAs, shRNAs, antisense RNAs, miRNAs, and ribozymes, dendrimers and aptamers; antibodies, including antibody fragments and intrabodies; and any combination thereof.

In some embodiments, the therapeutic agent is a chemotherapeutic agent. A “chemotherapeutic agent” is used to connote a compound or composition that is administered in the treatment of cancer. Chemotherapeutic agents useful in methods, compositions, and kits disclosed herein include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the presently disclosed biomimetic particles further comprise an imaging agent. In some embodiments, the imaging agent is labeled or conjugated with a fluorophore or radiotracer. Many appropriate imaging agents are known in the art, as are methods for their attachment to agents (e.g., attaching an imaging agent to a proteins or peptides using metal chelate complexes, radioisotopes, fluorescent markers, or enzymes whose presence can be detected using a colorimetric markers (such as, but not limited to, urease, alkaline phosphatase, (horseradish) hydrogen peroxidase and glucose oxidase). An agent may also be dual labeled with a radioisotope in order to combine imaging through nuclear approaches and be made into a unique cyclic structure and optimized for binding affinity and pharmacokinetics. Such agents can be administered by any number of methods known to those of ordinary skill in the art including, but not limited to, oral administration, inhalation, subcutaneous (sub-q), intravenous (I.V.), intraperitoneal (LP.), intramuscular (I.M.), or intrathecal injection. The methods, compositions, and kits described herein can be used alone or in combination with other techniques, to diagnose, access, monitor, and/or direct therapy. In some contexts, the imaging agent can be used for detecting and/or monitoring tumors or sites of metastasis in a patient. For example, a presently disclosed biomimetic particle can be administered in vivo and monitored using an appropriate label. Methods for detecting and/or monitoring a biomimetic particle labeled with an imaging agent in vivo include Gamma Scintigraphy, Positron Emission Tomography (PET), Single Photon Emission Computer Tomography (SPECT), Magnetic Resonance Imaging (MRI), X-ray, Computer Assisted X-ray Tomography (CT), Near Infrared Spectroscopy, and Ultrasound.

Many appropriate imaging agents are known in the art, such as paramagnetic ions (e.g., chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III)), radioactive isotopes (²¹¹astatine, ¹⁴carbon, ⁵¹chromium, ³⁶chlorine, ⁵⁷cobalt, ⁵⁸cobalt, ⁶⁷copper, ¹⁵²Eu, ⁶⁷gallium, ³hydrogen, ¹²³iodine, ¹²⁵iodine, ¹³¹iodine, ¹¹¹indium, ⁵⁹iron, ³²phosphorus, ¹⁸⁶rhenium, ¹⁸⁸rhenium, ⁷⁵selenium, ³⁵sulphur, ^{99m}technicium, ⁹⁰yttrium), fluorochromes (e.g., Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, Texas Red), PET and NMR-detectable substances (e.g., ⁶⁴Cu-ATSM, FDG, ¹⁸F-fluoride, FLT, FMISO, gallium, technetium-⁹⁹m, thallium), MRI imaging agents (e.g., gadolinium), X-ray imaging agents (barium, iodide), enzymes (urease, alkaline phosphatase, (horseradish) hydrogen peroxidase and glucose oxidase), secondary binding ligands (e.g., biotin and/or avidin and streptavidin), and azido group molecules (e.g., 2- and 8-azido analogues of purine nucleotides). In some contexts, an agent described herein can be employed in combination with a diagnostic agent.

In some embodiments, the targeting agent, therapeutic agent, and/or imaging agent is selected from the group consisting of a small molecule, carbohydrate, sugar, protein, peptide, nucleic acid, antibody or antibody fragment thereof, hormone, hormone receptor, receptor ligand, and cancer cell specific ligand. In some embodiments, a presently disclosed particle comprises more than one type of targeting agent, therapeutic agent, and/or imaging agent.

As used herein, the term “small molecule” refers to a molecule that contains several carbon-carbon bonds, and has a molecular weight of less than 5000 Daltons (5 kD), preferably less than 3 kD, still more preferably less than 2 kD, and most preferably less than 1 kD. In some cases it is preferred that a small molecule have a molecular weight equal to or less than 700 Daltons.

As used herein, an “RNA interference molecule” refers to an agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), microRNA (miRNA) and small molecules which interfere with or inhibit expression of a target gene by RNA interference (RNAi).

The term “polynucleotide” is used herein interchangeably with “nucleic acid” to indicate a polymer of nucleosides. Typically a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc.

As used herein, a “polymer” is a molecule of high relative molecule mass, the structure of which essentially comprises the multiple repetition of unit derived from molecules of low relative molecular mass, i.e., a monomer.

As used herein, the term “antibody” refers to a polypeptide or group of polypeptides which comprise at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as well as fragments, including Fab, Fab′, F(ab)₂, and F(ab′)₂ fragments.

In some embodiments, the biomimetic particle acts as an enhanced biomimetic artificial antigen presenting cell comprising one or more molecules capable of interacting with one or more T cell receptors on a T cell.

In some embodiments, the biomimetic particle is biodegradable and/or biocompatible in the patient. As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably do not induce inflammation or other adverse effects in vivo. As used herein, the term “biocompatible” means that the compound does not cause toxicity or adverse biological reaction in a patient when administered at a reasonable dose. Generally, to be biodegradable, the presently disclosed materials, e.g., microparticles and/or nanoparticles, contain a degradable linkage. Representative degradable linkages include, but are not limited to:

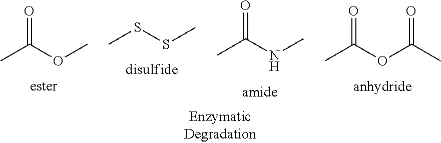

Non-limiting examples of biodegradable polymers include biodegradable poly-β-amino-esters (PBAEs), poly(amido amines), polyesters, polyanhydrides, bioreducible polymers, and other biodegradable polymers. Non-limiting examples of biodegradable polymers include poly(D,L-lactide-co-glycolide) (PLGA), poly (D,L-lactic acid) (PDLLA), polycaprolactone (PCL), polyglycolic acid (PGA), polylactic acid (PLA), poly(acrylic acid) (PAA), poly-3-hydroxybutyrate (P3HB) and poly(hydroxybutyrate-co-hydroxyvalerate). Other biodegradable polymers suitable for use with the presently disclosed subject matter are provided in U.S. Pat. No. 8,992,991, which is incorporated herein by reference in its entirety. In some embodiments, nondegradable polymers that are used in the art, such as polystyrene, are blended with a biodegradable polymer or polymers to form a presently disclosed particle. In some embodiments, a presently disclosed particle comprises GRAS (Generally Regarded As Safe) materials.

In some embodiments, the microparticle or nanoparticle comprises a polymeric matrix. Non-limiting examples of polymeric matrices include poly(lactic acid)-based polymeric matrices, such as polylactic acid (PLA), poly(D,L-lactide-co-glycolide) (PLGA), and poly (D,L-lactic acid) (PDLLA), as well as non-poly(lactic acid)-based polymeric matrices, such as polycaprolactone (PCL) and poly(beta-amino ester) (PBAE). In some embodiments, the polymeric matrix is selected from the group consisting of poly(D,L-lactide-co-glycolide) (PLGA), poly (D,L-lactic acid) (PDLLA), polycaprolactone (PCL), polyglycolic acid (PGA), polylactic acid (PLA), poly(acrylic acid) (PAA), poly-3-hydroxybutyrate (P3HB) and poly(hydroxybutyrate-co-hydroxyvalerate). In some embodiments, the polymeric matrix is polylactic acid (PLA). In some embodiments, the polymeric matrix is poly(D,L-lactide-co-glycolide) (PLGA). In some embodiments, the polymeric matrix is poly (D,L-lactic acid) (PDLLA). In some embodiments, the polymeric matrix is as polycaprolactone (PCL). In some embodiments, the polymeric matrix is poly(beta-amino ester) (PBAE). In some embodiments, the polymeric matrix is polyglycolic acid (PGA). In some embodiments, the polymeric matrix is poly(acrylic acid) (PAA). In some embodiments, the polymeric matrix is poly-3-hydroxybutyrate (P3HB). In some embodiments, the polymeric matrix is poly(hydroxybutyrate-co-hydroxyvalerate).

In some embodiments, blends of polymeric matrices may be used in the biomimetic particles, such as PLGA/PCL or PLGA/PBAE. In some embodiments, the PLGA content is about 50% to about 90%. In some embodiments, the PCL content is about 10% to about 50%. In some embodiments, the PBAE content is about 10% to about 50%. In some embodiments, the biomimetic particle further comprises a biodegradable polymer or blends of polymers blended with a nondegradable polymer.

II. Methods Comprising Biomimetic Particles

In some embodiments, the presently disclosed subject matter provides a method for delivering a drug to a diseased cell and/or tissue of a patient, the method comprising: (a) administering to a patient a biomimetic particle loaded with an agent for treating a disease or disorder, wherein the biomimetic particle is selected from the group consisting of: (i) a first biomimetic particle comprising: (1) a three-dimensional microparticle or nanoparticle; and (2) a naturally derived cell membrane, wherein the naturally derived cell membrane is not derived from a red blood cell; (ii) a second biomimetic particle comprising: (1) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (f) about 20 μm to about 100 μm; and (g) about 101 μm to about 1 mm; and (2) a naturally derived cell membrane; (iii) a third biomimetic particle comprising: (1) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (2) a naturally derived cell membrane; or (iv) a composition comprising (i), (ii), or (iii), or combinations of (i), (ii) and (iii); and (b) allowing the biomimetic particle to target a diseased cell and/or tissue of the patient, thereby delivering the drug to the diseased cell and/or tissue of the patient.

In some embodiments, the presently disclosed method further comprises stretching the presently disclosed particles to form anisotropic biomimetic particles. In some embodiments, the polymeric matrix is stretched from above the polymer transition temperature up to the polymer degradation temperature to form an anisotropic polymeric matrix. In some embodiments, the polymeric matrix is stretched at a temperature above but close to the polymer transition temperature to form an anisotropic polymeric matrix. Methods and devices for stretching can be found in Meyer et al. (J. Biomed. Mater. Res. A. 2015, 103(8):2747-57), which is incorporated herein by reference in its entirety.

For example, in some embodiments, if the polymer transition temperature is about 60° C., the polymeric matrix is stretched at a temperature above 60° C. to about 70° C. In some embodiments, if the polymer transition temperature is about 60° C., the polymeric matrix is stretched at a temperature above 60° C. to about 80° C. As used herein, the “polymer transition temperature” is the temperature range where the polymer transitions from a hard material to a soft or rubber-like material. As used herein, the “polymer degradation temperature” is the temperature where the polymer begins to disintegrate. In some embodiments, the polymeric matrix is stretched at a temperature from about 60° C. to about 90° C. to form an anisotropic polymeric matrix. In some embodiments, the polymeric matrix is stretched at a temperature of about 60° C. In some embodiments, stretching of the polymeric matrix causes the polymeric matrix to change from a generally spherical shape to an anisotropic shape.

In some embodiments, the presently disclosed subject matter provides a method for treating a disease or disorder in a patient in need thereof, the method comprising: (a) administering to a patient an effective amount of a biomimetic particle loaded with an agent for treating a disease or disorder, wherein the biomimetic particle is selected from the group consisting of: (i) a first biomimetic particle comprising: (1) a three-dimensional microparticle or nanoparticle; and (2) a naturally derived cell membrane, wherein the naturally derived cell membrane is not derived from a red blood cell; (ii) a second biomimetic particle comprising: (1) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (2) a naturally derived cell membrane; (iii) a third biomimetic particle comprising: (1) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (2) a naturally derived cell membrane; or (iv) a composition comprising (i), (ii), or (iii), or combinations of (i), (ii) and (iii); and (b) allowing the biomimetic particle to target a diseased cell and/or tissue of the patient, thereby treating the disease or disorder in the patient.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. In some embodiments, “treating” means prolonging survival of patients.

Non-limiting examples of diseases or disorders that can cause a diseased cell and/or tissue in a patient include blood diseases or disorders, inflammatory diseases, cancer, fibrotic diseases, autoimmune diseases, immune system disorders, infections, neurodegenerative diseases, cardiovascular diseases, hematological disorders, gastrointestinal diseases, liver diseases, endocrine system diseases, digestive disorders, musculoskeletal disorders, ocular disease, and respiratory disorders. In some embodiments, the disease or disorder that can be treated can be any disease that can be targeted by a presently disclosed particle comprising a drug to treat the disease or disorder. As is known in the art, the agent or agents that are loaded in the biomimetic particle will depend on the particular disease or disorder being treated.

In some embodiments, an agent does not need to be loaded into the biomimetic particle for the biomimetic particle to treat a disease or disorder. In some embodiments, the presently disclosed subject matter provides a method for treating a disease or disorder in a patient in need thereof, the method comprising administering to a patient an effective amount of a biomimetic particle for treating a disease or disorder, wherein the biomimetic particle is selected from the group consisting of: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c); thereby treating the disease or disorder in the patient.

For example, in some embodiments, the presently disclosed biomimetic particles treat a disease or disorder by acting as artificial red blood cells. In some embodiments, the particles may be used to treat a disease that affects the amount and/or function of red blood cells in a patient. As a non-limiting example, the particles may be used for treating thalassemia, which results from an abnormal formation of hemoglobin and requires repeated blood transfusions.

As another example, in some embodiments, the presently disclosed biomimetic particles treat a disease or disorder by acting as artificial platelets. In some embodiments, the particles may be used to treat a disease or disorder that affects the amount and/or function of platelets in a patient. Non-limiting examples of diseases or disorders that affect platelets include excessive bleeding, such as caused by trauma, thrombopenia, a disorder in which there is a relative decrease of platelets in the blood, thrombasthenia, a disorder with an abnormality of platelets, and a disorder causing sustained mechanical damage to platelets, such as occurs during extra corporeal circulation in coronary bypass surgery and/or haemodialysis.

In some embodiments, the disease or disorder is selected from the group consisting of excessive bleeding, thalassemia, thrombopenia, and thrombasthenia. In some embodiments, the disease or disorder is cancer. In some embodiments, the disease or disorder is an infectious disease. In some embodiments, the disease or disorder is a degenerative disease, such as a neurodegenerative disease. In some embodiments, the disease or disorder is excessive bleeding. In some embodiments, the disease or disorder is thalassemia. In some embodiments, the disease or disorder is thrombopenia. In some embodiments, the disease or disorder is thrombasthenia.

In some embodiments, the presently disclosed subject matter provides for the use of the presently disclosed compositions for the treatment of a disease or disorder. In some embodiments, the presently disclosed subject matter provides for the use of the presently disclosed biomimetic particles in the manufacture of a medicament for preventing, inhibiting, or treating diseases or disorders, such as those that affect the amount and/or function of red blood cells and/or platelets, such as excessive bleeding, thalassemia, thrombopenia, and thrombasthenia.

In some embodiments, the methods comprising the presently disclosed particles inhibit the disease or disorder by at least 5%, 10%, 15%, 20%, 25%, 30%, 33%, 35%, 40%, 45%, 50%, 55%, 60%, 66%, 70%, 75%, 80%, 85%, 90%, 91%, 92%, 93%, 94%, 95%, 96%, 97%, 98%, 99% or more as compared to the extent of the disease or disorder in the patient when the methods comprising the presently disclosed particles are not used.

In some embodiments, methods comprising the presently disclosed particles extend survival of the patient. For example, the presently disclosed methods can extend survival (e.g., progression free survival) of the patient by 5%, 10%, 15%, 20%, 25%, 30%, 33%, 35%, 40%, 45%, 50%, 55%, 60%, 66%, 70%, 75%, 80%, 85%, 90%, 91%, 92%, 93%, 94%, 95%, 96%, 97%, 98%, 99%, 1-fold, 1.1-fold, 1.2-fold, 1.3-fold, 1.4-fold, 1.5-fold, 1.6-fold, 1.7-fold, 1.8-fold, 1.9-fold, 2.0 fold, 2.5-fold, 3.0-fold, 3.5-fold, 4.0-fold, 5.0-fold, 6-fold, 7-fold, 8-fold, 9-fold, 10-fold, or more as compared to survival of the patient when the presently disclosed methods are not used. In some embodiments, the survival is progression-free survival.

As used herein, the term “reduce” or “inhibit,” and grammatical derivations thereof, refers to the ability of an agent to block, partially block, interfere, decrease, reduce or deactivate a biological molecule, pathway or mechanism of action. Thus, one of ordinary skill in the art would appreciate that the term “inhibit” encompasses a complete and/or partial loss of activity, e.g., a loss in activity by at least 10%, in some embodiments, a loss in activity by at least 20%, 30%, 50%, 75%, 95%, 98%, and up to and including 100%.

In some embodiments, the presently disclosed subject matter provides a method for imaging a diseased cell and/or tissue of a patient, the method comprising: (a) administering to a patient a biomimetic particle comprising: (i) a three-dimensional microparticle or nanoparticle and a naturally derived cell membrane, wherein the naturally derived cell membrane is not derived from a red blood cell; or (ii) a three-dimensional microparticle or nanoparticle having an asymmetrical shape, wherein the asymmetrical shape has at least one surface having a radius of curvature along at least one axis selected from one of the following ranges: (1) about 1 nm to about 10 nm; (2) about 11 nm to about 100 nm; (3) about 101 nm to about 400 nm; (4) about 401 nm to about 1 μm; (5) about 10 μm to about 20 μm; (6) about 20 μm to about 100 μm; and (7) about 101 μm to about 1 mm; and a naturally derived cell membrane; (iii) an imaging agent; and (iv) a targeting agent; wherein upon administration, the biomimetic particle targets a diseased cell and/or tissue of the patient; and (b) imaging the diseased cell and/or tissue of the patient.

In some embodiments, imaging the diseased cell and/or tissue of the patient occurs by bioluminescent imaging, fluorescent imaging, x-ray computed tomography (CT), magnetic resonance imaging (MRI), positron emission tomography (PET), single-photon emission computed tomography (SPECT), and/or X-ray, and operable combinations thereof.

In some embodiments, the presently disclosed subject matter provides a method for administering a biotinylated drug to a patient, the method comprising administering to a patient a biomimetic particle conjugated with a biotin-binding protein or fragment thereof, wherein the biomimetic particle is selected from the group consisting of: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c).

Biotin has been found to have a high affinity for biotin-binding proteins, such as avidin, streptavidin, and neutravidin. Biotinylation is the process of covalently attaching biotin to a protein, antibody, enzyme, nucleic acid or other molecule. Biotinylated molecules can comprise multiple biotin molecules. In some embodiments, these biotinylated molecules can bind to biotin-binding proteins on the presently disclosed non-spherical artificial cells. Accordingly, in some embodiments, the protein or fragment thereof is a biotin-binding protein or fragment thereof. In some embodiments, the biotin-binding protein or fragment thereof is selected from the group consisting of avidin, streptavidin, neutravidin, and fragment thereof. In some embodiments, the biotinylated drug is a biotinylated antibody. In some embodiments, the biotinylated antibody is specific for CD28, a co-stimulatory surface protein in the activation of lymphocytes. As used herein, a “drug” is a substance that has a physiological effect when introduced into a subject. In some embodiments, more than one kind of drug is loaded into a presently disclosed particle, such that a drug mixture can be simultaneously administered to the patient.

The terms “subject” and “patient” are used interchangeably herein. The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease.

Generally, the presently disclosed biomimetic particles can be administered to a subject for therapy by any suitable route of administration, including orally, nasally, transmucosally, ocularly, rectally, intravaginally, or parenterally, including intravenous, intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

The phrases “systemic administration”, “administered systemically”, “peripheral administration” and “administered peripherally” as used herein mean the administration of compositions such that they enter the patient's system and, thus, are subject to metabolism and other like processes, for example, subcutaneous or intravenous administration.

The phrases “parenteral administration” and “administered parenterally” as used herein mean modes of administration other than enteral and topical administration, usually by injection, and includes, without limitation, intravenous, intramuscular, intarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection and infusion.

The presently disclosed pharmaceutical compositions can be manufactured in a manner known in the art, e.g. by means of conventional mixing, dissolving, granulating, dragee-making, levitating, emulsifying, encapsulating, entrapping or lyophilizing processes.

In another embodiment, the presently disclosed pharmaceutical compositions may comprise PEGylated therapeutics (e.g., PEGylated antibodies). PEGylation is a well-established and validated approach for the modification of a range of antibodies, proteins, and peptides and involves the attachment of polyethylene glycol (PEG) at specific sites of the antibodies, proteins, and peptides (Chapman (2002) Adv. Drug Deliv. Rev. 54:531-545). Some effects of PEGylation include: (a) markedly improved circulating half-lives in vivo due to either evasion of renal clearance as a result of the polymer increasing the apparent size of the molecule to above the glomerular filtration limit, and/or through evasion of cellular clearance mechanisms; (b) improved pharmacokinetics; (c) improved solubility—PEG has been found to be soluble in many different solvents, ranging from water to many organic solvents such as toluene, methylene chloride, ethanol and acetone; (d) PEGylated antibody fragments can be concentrated to 200 mg/ml, and the ability to do so opens up formulation and dosing options such as subcutaneous administration of a high protein dose; this is in contrast to many other therapeutic antibodies which are typically administered intravenously; (e) enhanced proteolytic resistance of the conjugated protein (Cunningham-Rundles et. al. (1992) J. Immunol. Meth. 152:177-190); (0 improved bioavailability via reduced losses at subcutaneous injection sites; (g) reduced toxicity has been observed; for agents where toxicity is related to peak plasma level, a flatter pharmacokinetic profile achieved by sub-cutaneous administration of PEGylated protein is advantageous; proteins that elicit an immune response which has toxicity consequences may also benefit as a result of PEGylation; and (h) improved thermal and mechanical stability of the PEGylated molecule.

Pharmaceutical compositions for parenteral administration include aqueous solutions of compositions. For injection, the presently disclosed pharmaceutical compositions can be formulated in aqueous solutions, for example, in some embodiments, in physiologically compatible buffers, such as Hank's solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of compositions include fatty oils, such as sesame oil, or synthetic fatty acid esters, such as ethyl oleate or triglycerides, or liposomes. Optionally, the suspension also can contain suitable stabilizers or agents that increase the solubility of the compositions to allow for the preparation of highly concentrated solutions.

For nasal or transmucosal administration generally, penetrants appropriate to the particular barrier to be permeated are used in the formulation. Such penetrants are generally known in the art.

For inhalation delivery, the agents of the disclosure also can be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Additional ingredients can be added to compositions for topical administration, as long as such ingredients are pharmaceutically acceptable and not deleterious to the epithelial cells or their function. Further, such additional ingredients should not adversely affect the epithelial penetration efficiency of the composition, and should not cause deterioration in the stability of the composition. For example, fragrances, opacifiers, antioxidants, gelling agents, stabilizers, surfactants, emollients, coloring agents, preservatives, buffering agents, and the like can be present. The pH of the presently disclosed topical composition can be adjusted to a physiologically acceptable range of from about 6.0 to about 9.0 by adding buffering agents thereto such that the composition is physiologically compatible with a subject's skin.

Regardless of the route of administration selected, the presently disclosed compositions can be formulated into pharmaceutically acceptable dosage forms such as described herein or by other conventional methods known to those of skill in the art.

In general, the “effective amount” or “therapeutically effective amount” of an active agent refers to the amount necessary to elicit the desired biological response. As will be appreciated by those of ordinary skill in this art, the effective amount of an agent may vary depending on such factors as the desired biological endpoint, the agent to be delivered, the composition of the encapsulating matrix, the target tissue, and the like.

The term “combination” is used in its broadest sense and means that a subject is administered at least two agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In some embodiments of the presently disclosed subject matter, the active agents are combined and administered in a single dosage form. In some embodiments, the active agents are administered in separate dosage forms (e.g., wherein it is desirable to vary the amount of one but not the other). The single dosage form may include additional active agents for the treatment of the disease state.

Further, the presently disclosed compositions can be administered alone or in combination with adjuvants that enhance stability of the agents, facilitate administration of pharmaceutical compositions containing them in certain embodiments, provide increased dissolution or dispersion, increase activity, provide adjuvant therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of two (or more) agents can be varied so long as the beneficial effects of the combination of these agents are achieved. Accordingly, the phrase “in combination with” refers to the administration of a presently disclosed composition and, optionally, additional agents either simultaneously, sequentially, or a combination thereof. Therefore, a subject administered a combination of a presently disclosed composition and, optionally, additional agents can receive a presently disclosed composition and, optionally, additional agents at the same time (i.e., simultaneously) or at different times (i.e., sequentially, in either order, on the same day or on different days), so long as the effect of the combination of all agents is achieved in the subject.

When administered sequentially, the agents can be administered within 1, 5, 10, 30, 60, 120, 180, 240 minutes or longer of one another. In other embodiments, agents administered sequentially, can be administered within 1, 2, 3, 4, 5, 10, 15, 20 or more days of one another. Where the agents are administered simultaneously, they can be administered to the subject as separate pharmaceutical compositions, each comprising either a presently disclosed composition and, optionally, additional agents, or they can be administered to a subject as a single pharmaceutical composition comprising all agents. In some embodiments, one agent is administered and the other agent is administered three days later. In some embodiments, one agent is administered and the other agent is administered 4, 5, 6, 7, 8, 9, 10, 15, 20 days or more later.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an agent and at least one additional therapeutic agent is greater than the sum of the biological activities of the respective agents when administered individually.

Synergy can be expressed in terms of a “Synergy Index (SI),” which generally can be determined by the method described by F. C. Kull et al. Applied Microbiology 9, 538 (1961), from the ratio determined by:

QₐQ_{A}+Q_{b}Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In another aspect, the presently disclosed subject matter provides a pharmaceutical composition and optionally, additional agents, alone or in combination with one or more additional therapeutic agents in admixture with a pharmaceutically acceptable excipient.

More particularly, the presently disclosed subject matter provides a pharmaceutical composition and, optionally, additional agents and a pharmaceutically acceptable carrier.

In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams and Wilkins (2000).

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

III. Kits Comprising Biomimetic Particles

In general, a presently disclosed kit contains some or all of the components, reagents, supplies, and the like to practice a method according to the presently disclosed subject matter. In some embodiments, the term “kit” refers to any intended article of manufacture (e.g., a package or a container) comprising a presently disclosed biomimetic particle formulation.

In some embodiments, the presently disclosed subject matter provides a kit comprising the biomimetic particle or composition selected from the group consisting: (a) a first biomimetic particle comprising: (i) a three-dimensional microparticle or nanoparticle; and (ii) a naturally derived cell membrane, wherein the naturally derived cell membrane is not derived from a red blood cell; (b) a second biomimetic particle comprising: (i) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (f) about 20 μm to about 100 μm; and (g) about 101 μm to about 1 mm; and (ii) a naturally derived cell membrane; (c) a third biomimetic particle comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a naturally derived cell membrane; or (d) a composition comprising (a), (b), or (c), or combinations of (a), (b) and (c).

In some embodiments, the kit can be packaged in a divided or undivided container, such as a carton, bottle, ampule, tube, etc. The presently disclosed compositions can be packaged in dried, lyophilized, or liquid form. Additional components provided can include vehicles for reconstitution of dried components. Preferably all such vehicles are sterile and apyrogenic so that they are suitable for injection into a patient without causing adverse reactions.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

### Particle Preparation and Characterization

All particles synthesized for this study were fabricated into microspheres by a single emulsion technique for organic polymeric particles. 100 mg of a chosen polymer or polymer blend was dissolved in 5 mL of dichloromethane. To generate labeled particles which could be visible across multiple fluorescence channels, 7-amino-4-methyl coumarin, (Sigma Aldrich; St. Louis, Mo.), coumarin-6 (Sigma Aldrich; St. Louis, Mo.), and Nile Red (Life Technologies; Grand Island, N.Y.) dyes were added to the DCM each at a 1% w/w ratio to the polymer. The polymer-DCM solution was homogenized into 50 mL of a 1% PVA solution for one minute at either a low speed of 5,000 rpm, medium speed of 10,000 rpm, high speed of 15,000 rpm, or for nanoparticle synthesis, sonication at 12 W for 120 seconds, to generate the particles used in this study. The homogenizer low speed of 5,000 rpm was selected as the standard formulation condition for the standard sized particles utilized most often in this study. The initial emulsion was then poured into 100 mL of a 0.5% PVA solution and the DCM was allowed to evaporate over the course of 4 hours to permit for the formation of particles. The sample was then washed three times with water to yield the final product which was frozen and lyophilized prior to use in thin film stretching studies.

Most polymeric biomaterials utilized in this study were purchased commercially including the PLGA 50:50 lactic acid to glycolic acid content, MW 38,000-54,000 Da (Sigma-Aldrich; St. Louis, Mo.) and the PCL (MW 80,000 Da) (Sigma Aldrich; St. Louis, Mo.). The poly(beta-amino ester)s (PBAE)s utilized in this study were synthesized from commercially available monomers as described previously with modifications (Little et al. 2004). Briefly 1, 4 butanediol diacrylate (Alfa Aesar; Ward Hill, Mass.) and 4, 4′-trimethylenedipiperidine (Sigma Aldrich; St. Louis, Mo.) were dissolved together in a 1.05:1 molar ratio in 5 mL of dichloromethane and heated at 90° C. for 24 hrs. The resulting polymer was then reacted and end-capped at room temperature for one hour with 1-(3-Aminopropyl)-4-methylpiperazine (Alfa Aesar; Ward Hill, Mass.) in a 10-fold molar excess. The final polymer was then purified by addition of hexane, and the precipitated polymer was dried under a vacuum for 2 days. The polymer was then resuspended in dichloromethane at 100 mg/mL and stored in a dry environment at −20° C. until use.

### Thin Film Stretching Method

The thin film stretching method adapted for this study was originally developed by Ho et al. (1993) and recently expanded by Champion et al. (2007) to produce particles of anisotropic shape. The lyophilized particles were suspended in 1 mL of water and then mixed with 19 mL of a 10% w/w PVA and 2% w/w glycerol solution by trituration. The resulting particle solution was then cast into films in 5 mL aliquots onto 5 cm×7 cm rectangular petri dishes (VWR International; Radnor, Pa.) for 1D stretching or in 10 mL aliquots onto 10 cm×10 cm (Thermo Fisher; Rockville, Md.) square petri dishes for 2D stretching. The films were allowed to dry overnight and were then removed from the petri dish. The film was cut to size and then mounted on the aluminum blocks and heated to 70° C. unless otherwise noted to bring the polymeric microparticles above their glass transition temperatures. After 10 min of heating, the program was loaded to the microcontroller and the stepper motors were instructed to pull apart the film at a strain rate of 0.2 min⁻¹ (unless otherwise noted). The film was then allowed to rest for 1 min and then removed from the oven and allowed to cool for 20 min. After cooling, the film was cut from the grips and dissolved in 25 mL of water. The resulting particle suspension was then washed 3 times and lyophilized prior to use.

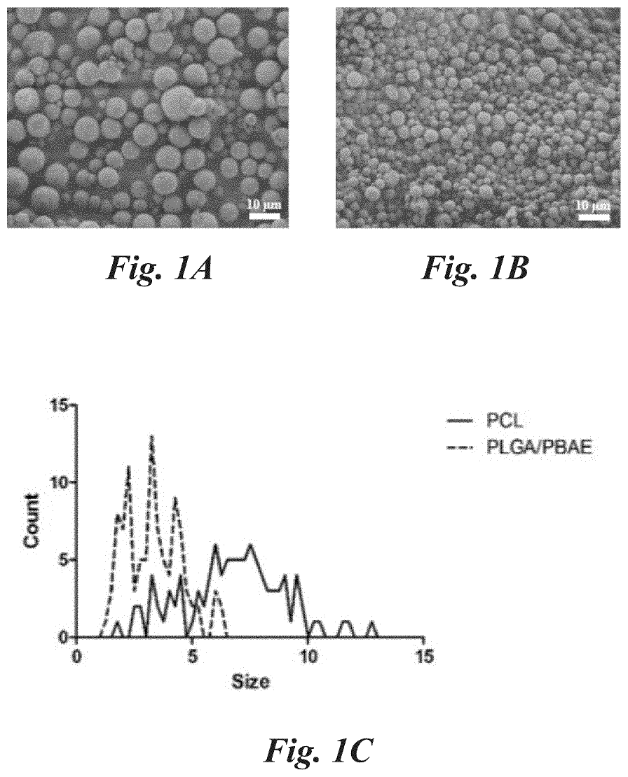

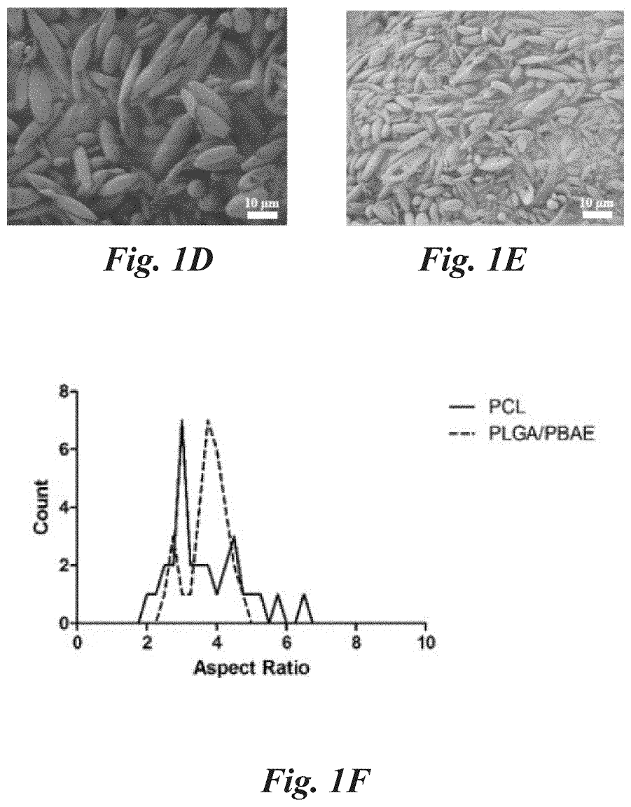

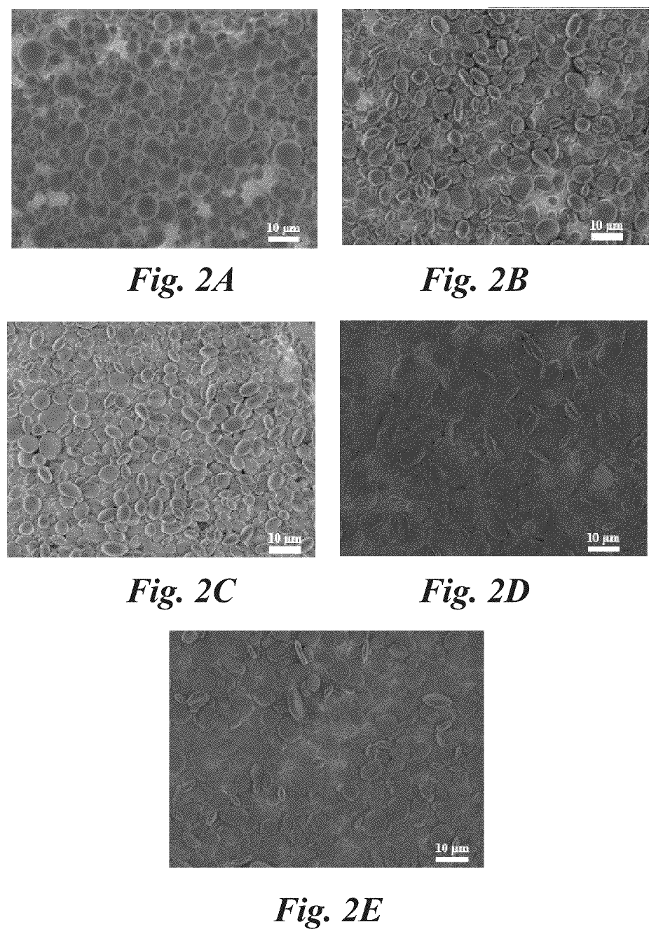

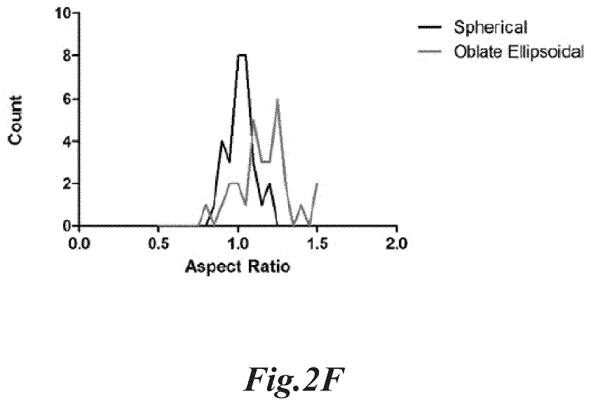

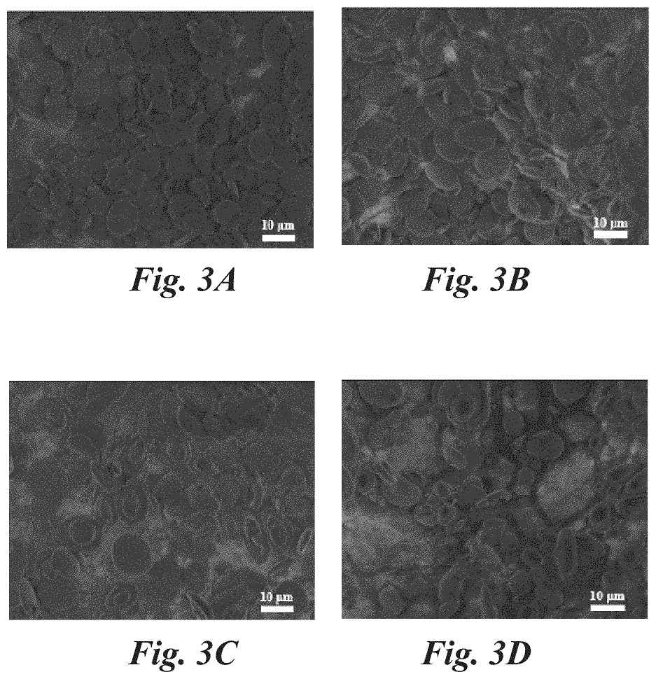

All imaging was conducted with a Leo FESEM scanning electron microscope. To prepare samples for analysis, lyophilized particles were spread onto carbon tape (Nisshin EM Co.; Tokyo, Japan) adhered to aluminum tacks (Electron Microscopy Services; Hatfield, Pa.). The excess particles were removed and the particles then were sputter coated with a 20 nm thick layer of gold-palladium. The samples were then loaded into the microscope and imaged. All images were processed in ImageJ to obtain relevant measurements (size, aspect ratio, etc.). Representative particles that can be fabricated using this method are given in FIG. 1A, FIG. 1B, FIG. 1C, FIG. 1D, FIG. 1E, FIG. 1F, FIG. 2A, FIG. 2B, FIG. 2C, FIG. 2D, FIG. 2E, FIG. 2F, FIG. 3A, FIG. 3B, FIG. 3C, and FIG. 3D.

### Biomimetic Functional Membrane Preparation

### Membrane Preparation 1—RBC

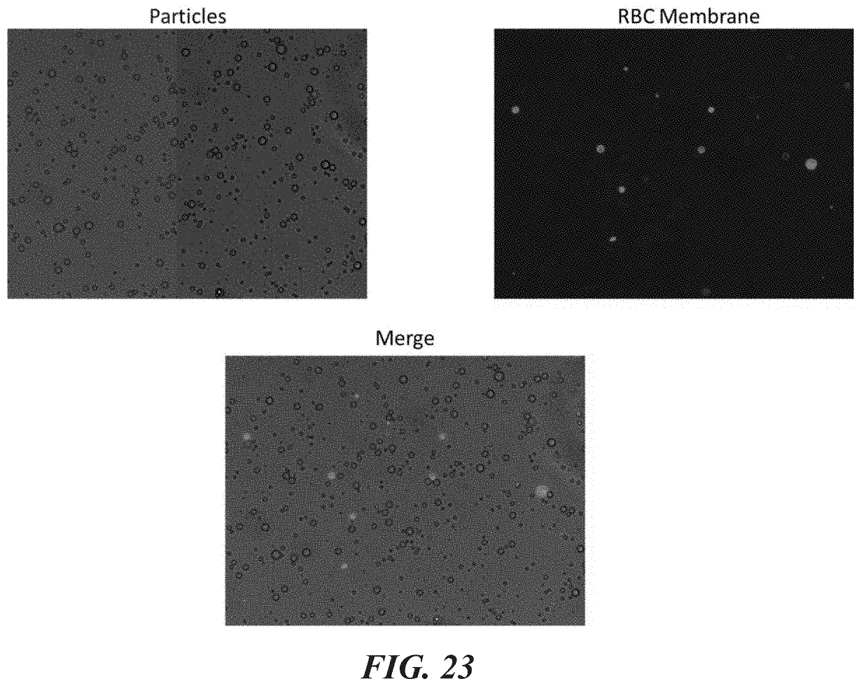

Biomimetic red blood cell or platelet membranes were fused with particles based on a previously established protocol for biomimetic particle coatings with modification (Hu et al., 2011). Briefly, 500 μL total whole blood was collected from black 6 mice or human sources and centrifuged at 100 g for 5 min to separate out blood into cellular components. For red blood cell processing, the plasma and buffy coat was removed and the remaining RBCs were suspended in 1 mL of 1×PBS. The RBCs were centrifuged again at 100 g for 5 min and then they were resuspended in 0.25×PBS for hypotonic lysis for 20 min at 4° C. The resulting RBC ghosts were centrifuged at 800 g for 5 min and then resuspended in 1 mL PBS. The RBC ghosts were stained for visualization with DiD dye for 30 min at 37° C. After washing 3 times at 5,000 g for 5 min with 1 mL of 1×PBS, the RBC ghosts were processed into submicron vesicles through 2 min of sonication in a VCX500 20 kHz sonicator using a cup horn at an amplitude of 60%. Functionalized lipids including maleimide active lipid were added prior to sonication if desired for custom tailoring of membrane presented ligands. The resulting submicron vesicles were then further processed into 200 nm vesicles using an Avanti mini extruder with a 200 nm membrane filter. 200 μL of this vesicle suspension was added to 5 mg of particles of desired shape and suspended in 1 mL of water and sonicated for 30 seconds at 20% amplitude using a VCX500 sonicator equipped with a microtip. After washing 3 times in water, the particles were imaged and utilized for the desired application. Of note, it was previously known within the art that to prepare supported biomimetic membranes on spherical particles, an extrusion process was used. However, it was found that an extrusion process was not suitable to prepare supported biomimetic membranes on anisotropic particles due to non-uniform coating, as shown in FIG. 23. The co-sonication method presented herein overcomes the issue of uniform membrane coating.

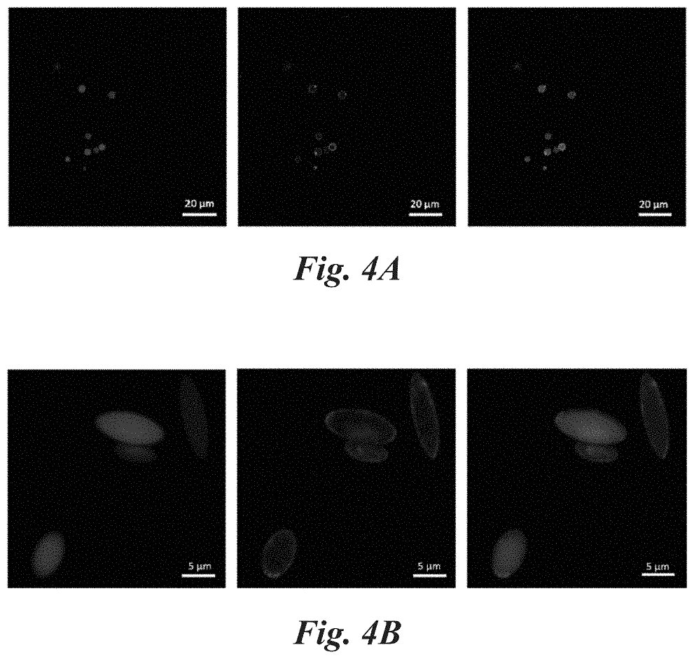

To analyze the formation of supported biomimetic membranes, confocal imaging of PLGA particles encapsulating 7-AMC were coated with DiD containing membrane derived vesicles. A representative image is shown in FIG. 4A and FIG. 4B. Confocal image acquisition was completed with a Zeiss 780 FCS Confocal Microscope. To derive profile information, we used the ImageJ profile measurement tool and drew a line through the particle to determine relative fluorescence information.

### Membrane Preparation 2—RBC, Platelet (Resting & Activated), and Dendritic Cell

Biomimetic red blood cell or platelet membranes were fused with particles based on a previously established protocol for biomimetic particle coatings with modification (Hu et al., 2011). Human whole blood and platelet concentrate (ZenBio Inc.; Durham, N.C.) were processed to generate red blood cell and platelet vesicles, respectively. For red blood cell processing, whole blood was aliquoted into 1 mL samples and centrifuged at 800 g for 5 min to separate out blood into cellular components. The plasma and buffy coat were removed and the remaining RBCs were suspended in 1 mL of 1×PBS. The RBCs were centrifuged again at 800 g for 5 min and then resuspended in 0.25×PBS for hypotonic lysis for 20 min at 4° C. The resulting RBC ghosts were centrifuged at 17,000 g for 5 min and then resuspended in 1 mL PBS. For platelet processing, platelets were suspended in 1 mM EDTA buffer and centrifuged at 500 g for 10 min. Supernatant was collected and washed twice by centrifugation at 4000 g for 20 min in EDTA buffer. Platelets were adjusted to concentration of 2×10⁹ cells/mL and stored at −80° C. For activated platelet processing, platelets were activated with 10 μM adenosine 5′-diphosphate (ADP) (Sigma-Aldrich; St. Louis, Mo.) in EDTA buffer to prevent aggregation prior to freezing. Resting or activated platelets were thawed at room temperature to disrupt membranes and 300 μL was suspended in EDTA buffer and washed 3 times by centrifugation at 4000 g for 3 min in 1×PBS.

Red blood cell, resting platelet, and activated platelet membranes were then processed to generate fluorescent submicron vesicles. 50 μg of DOPC-PEG and 50 μg of DSPE-Rhodamine (Avanti Polar Lipids; Alabaster, Ala.) were mixed in a scintillation vial and dried in the desiccator. 1 mL of processed ghosts were then added to the dried fluorescent lipids and vortexed to resuspend lipids. The ghosts were incubated for 1 hour at 37° C. on an orbital mixer. After washing 3 times at 1,000 g for 5 min with 1 mL of 1×PBS, the ghosts were processed into submicron vesicles through 2 min of sonication in a VCX500 20 kHz sonicator using a cup horn at an amplitude of 50%. Activated platelet ghosts were then washed at 1000 g for 5 min to remove debris. PLGA micro- or nanoparticles were then coated with ghosts. 2 mg of particles were added to 0.5 mL of ghosts and 0.5 mL of 1×PBS and sonicated in a VCX500 20 kHz sonicator using a cup horn at an amplitude of 50%. The particles were washed 3 times by centrifugation for 5 min at 1000 g for microparticles and 10,000 g for nanoparticles.

Dendritic cell membrane vesicles were prepared in a similar fashion to a procedure previously reported (Fang et al., 2015). Briefly a confluent T-175 flask of the dendritic cell line DC 2.4 was detached from a flask and washed 3 times with PBS. The cells were then suspended in 1 mL ACK lysis buffer and homogenized with a 2 mL Dounce homogenizer using a tight-fitting pistil. The cell lysate was spun at 3200 g for 5 min to remove intact cellular and nuclear material and the supernatant was reserved. Then the pellet was resuspended in 1 mL ACK lysis buffer and homogenized again. The second cell lysate was spun out again and the supernatant was pooled with the supernatant collected from the previous spin. The pooled supernatants were then spun out at 17000 g for 20 min to remove subcellular organelles. The supernatants of this spin were then collected and spun once more at 100,000 g for 1 hr. to remove the membrane vesicle fraction. These vesicles were then resuspended in PBS and mixed with 1 mg of PLGA for the membrane coating.

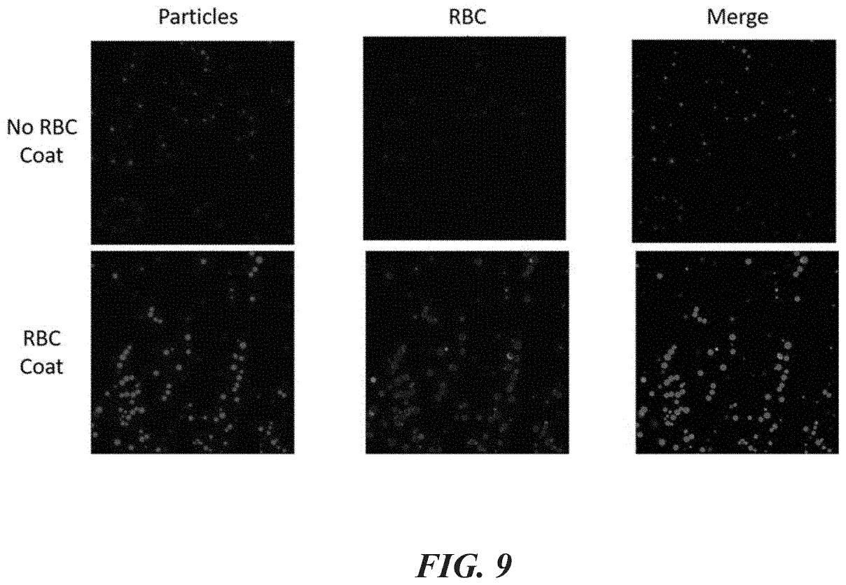

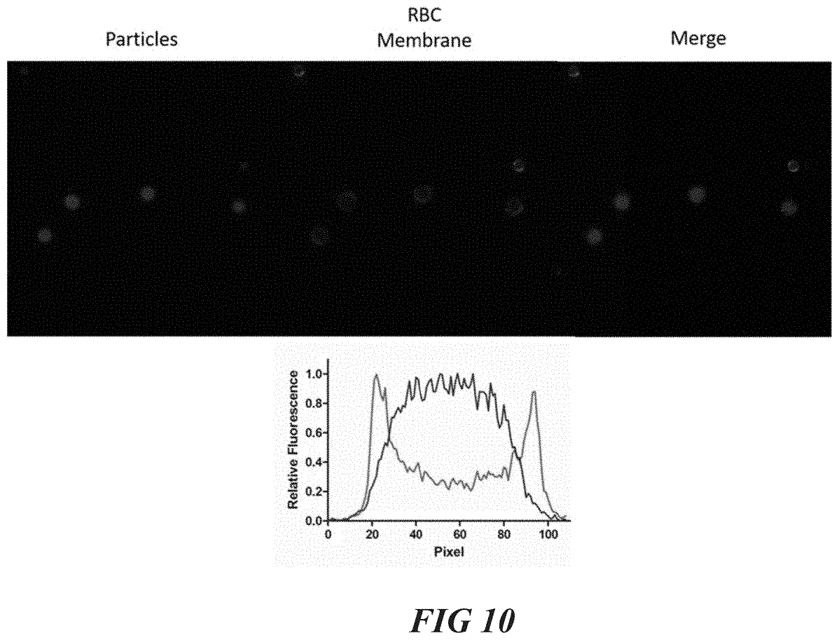

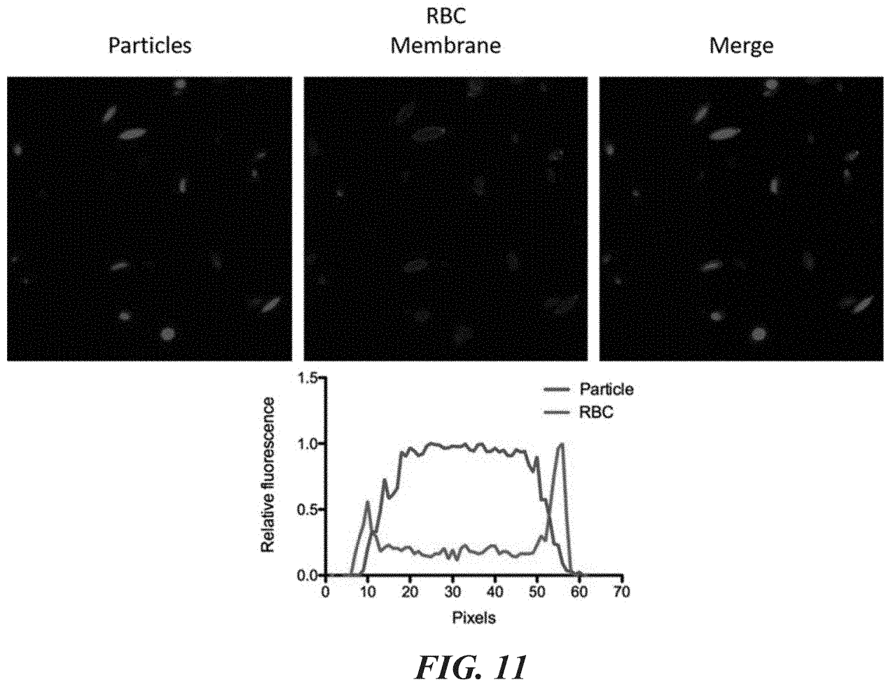

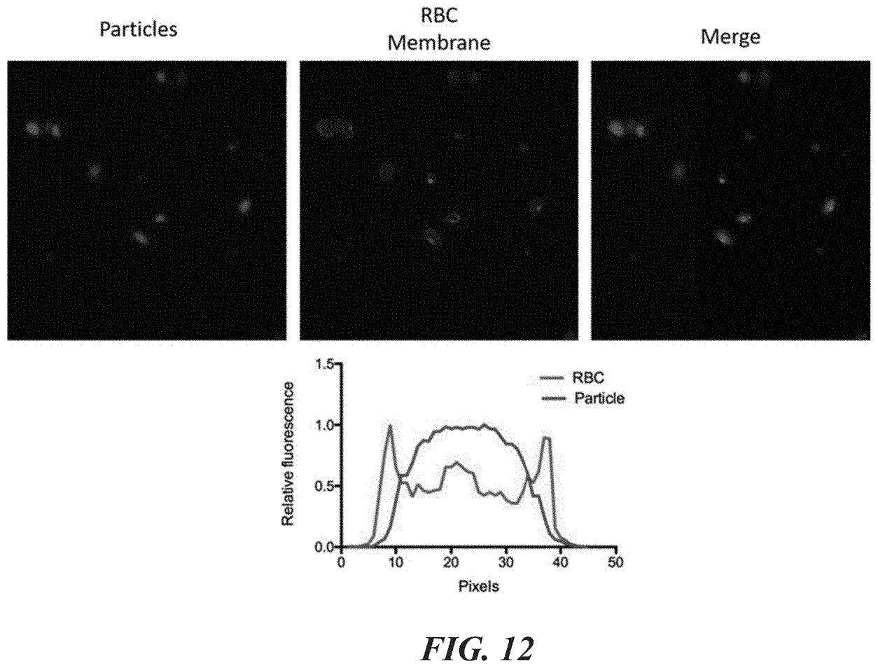

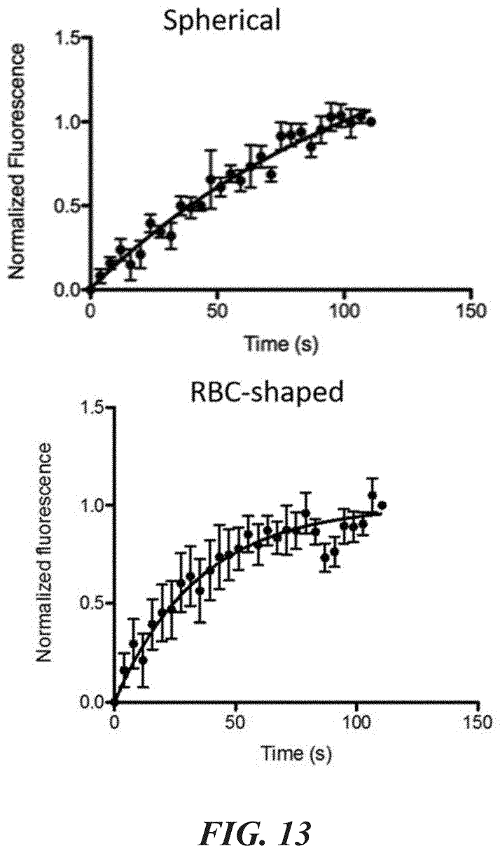

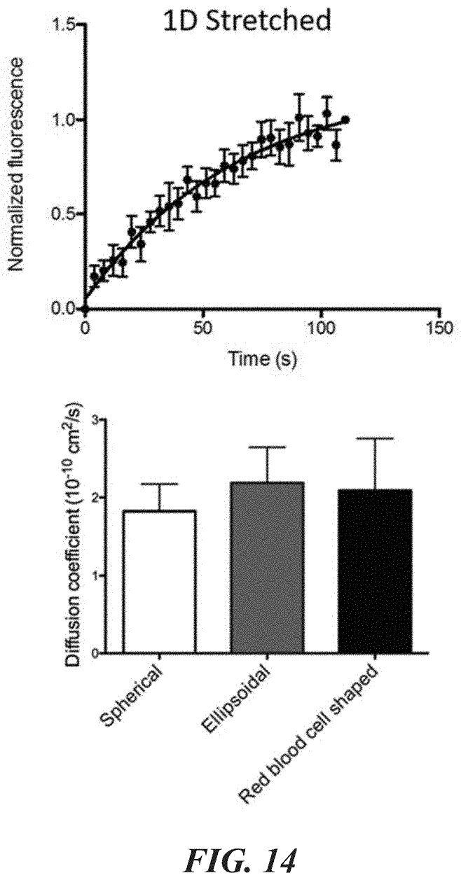

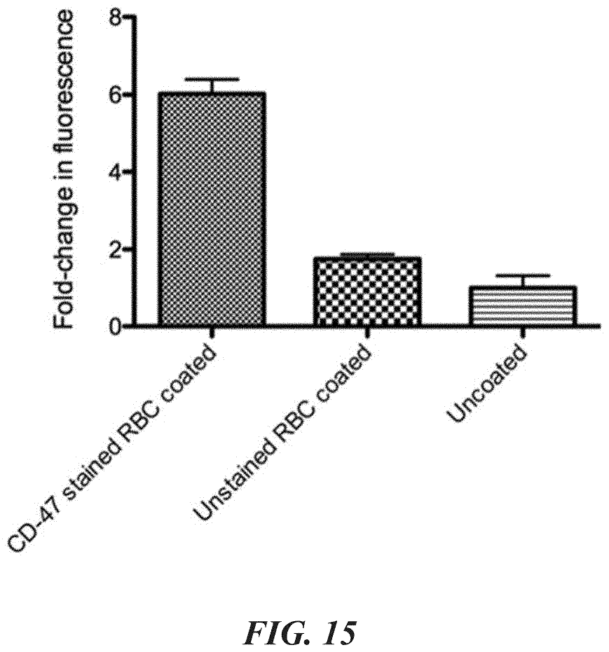

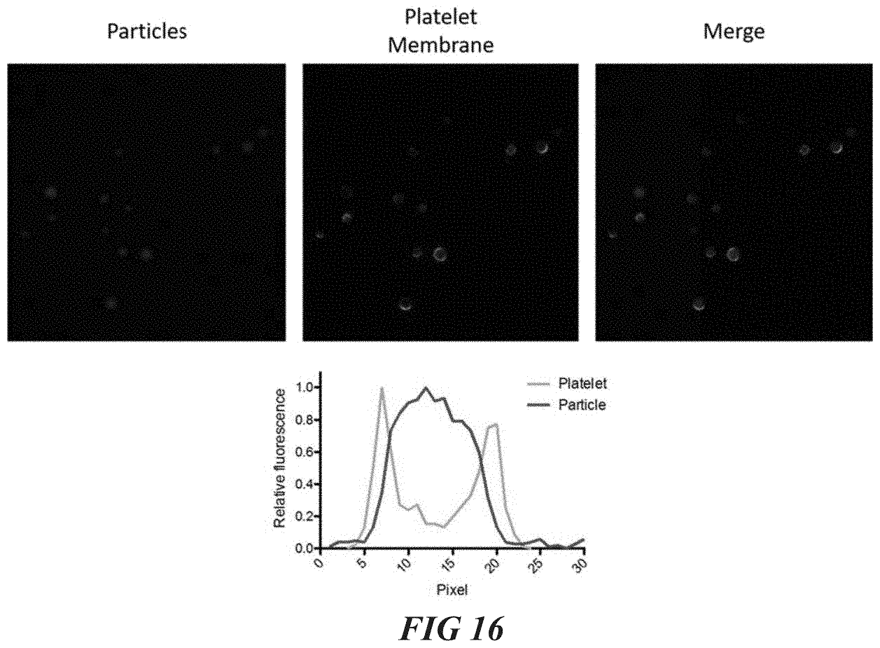

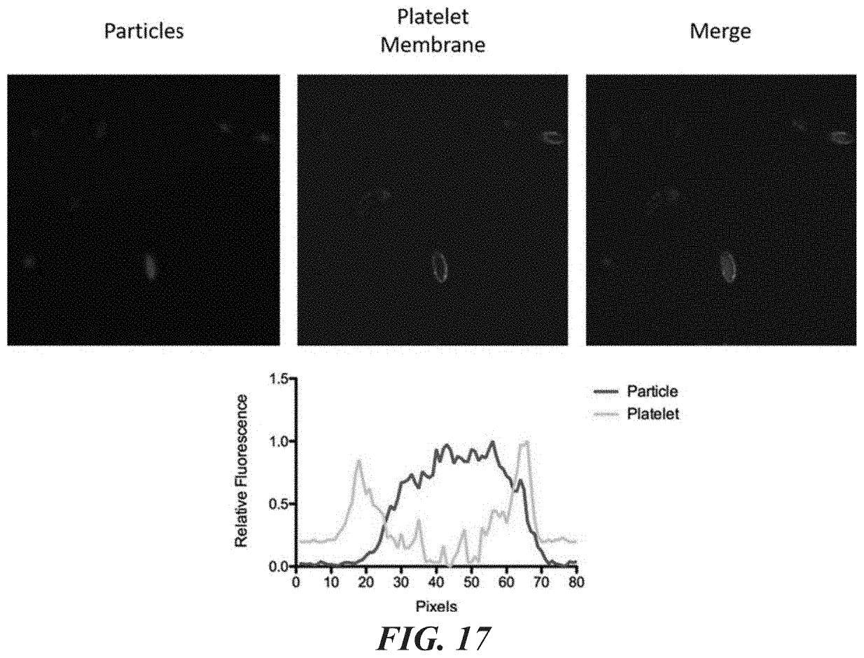

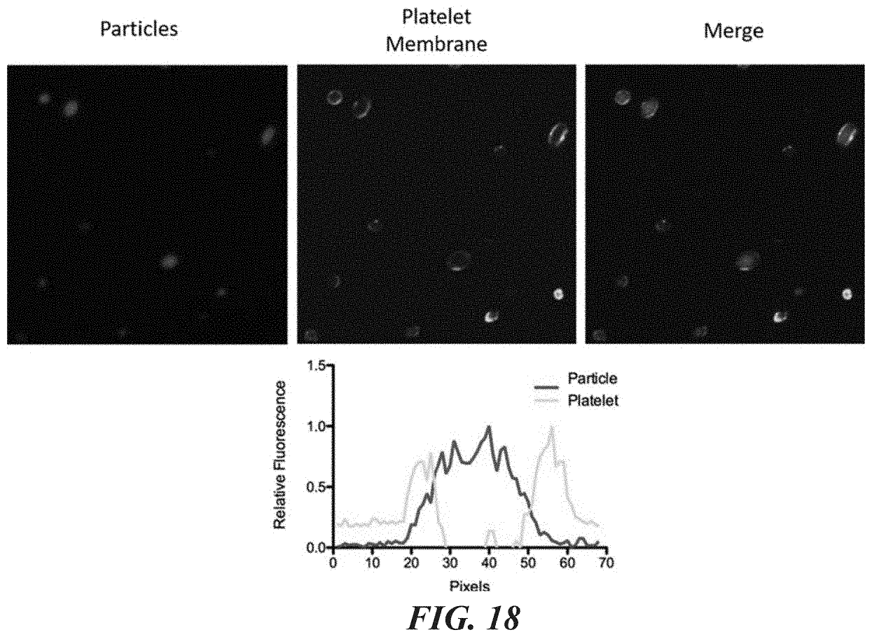

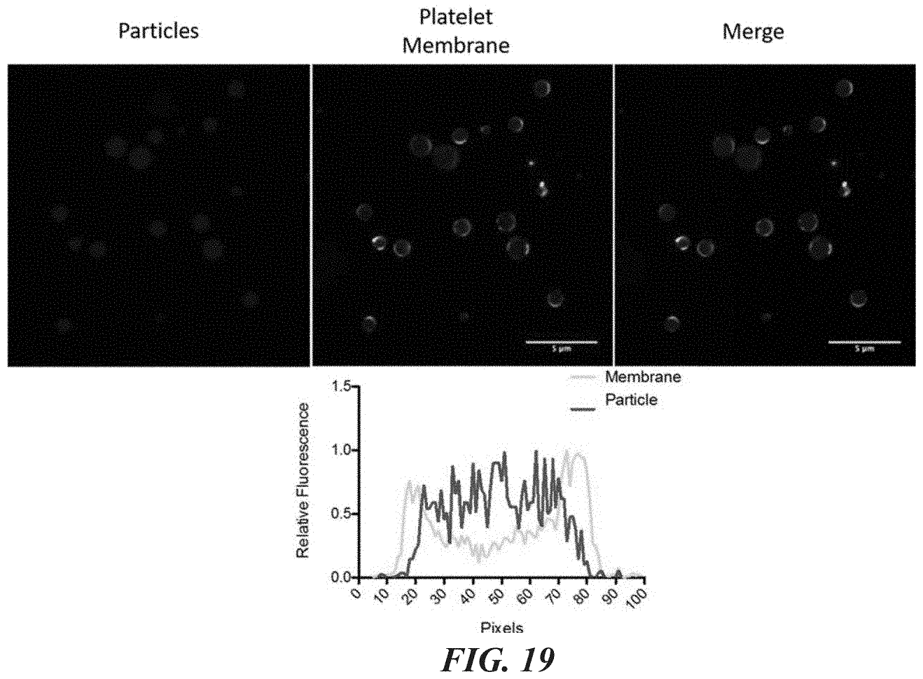

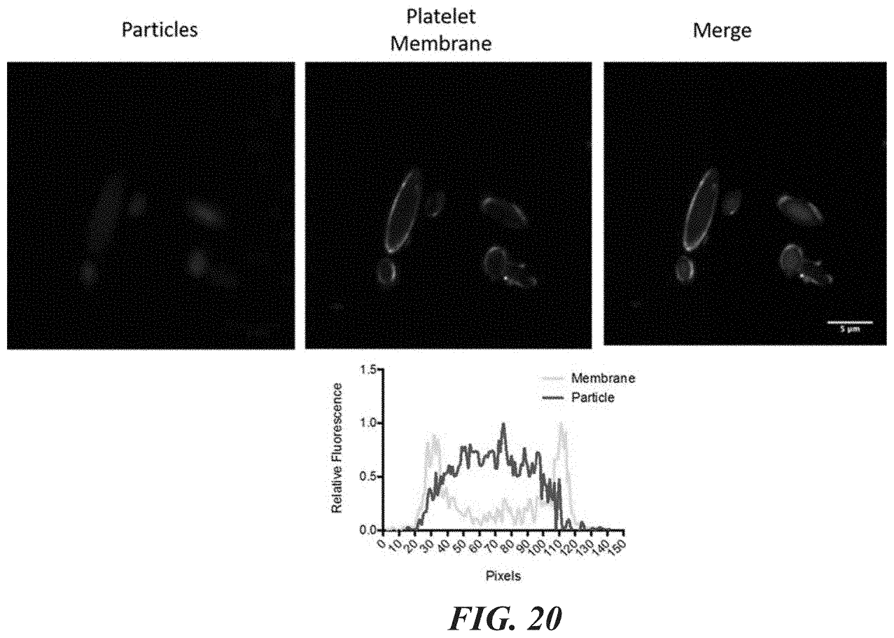

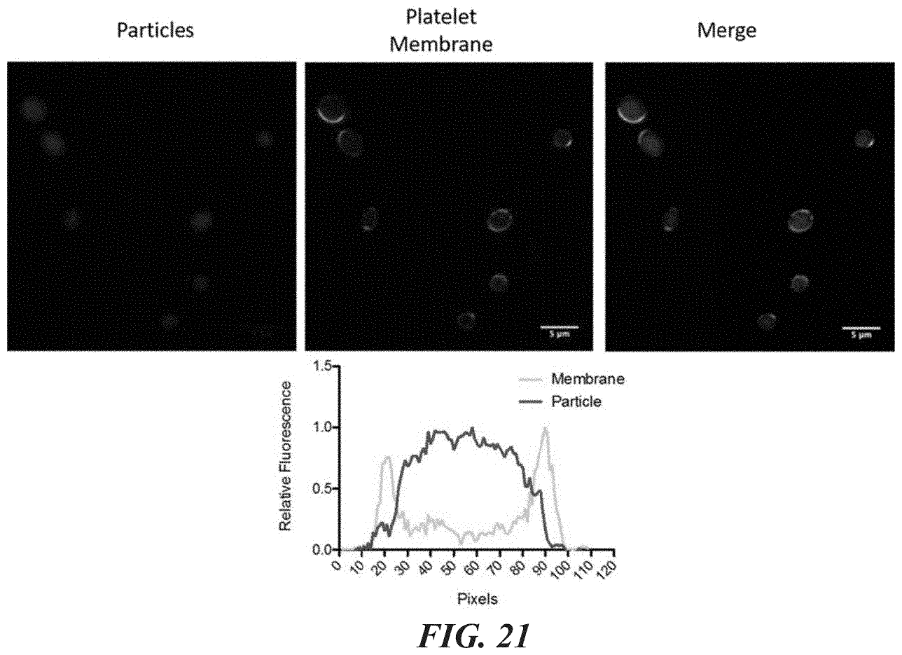

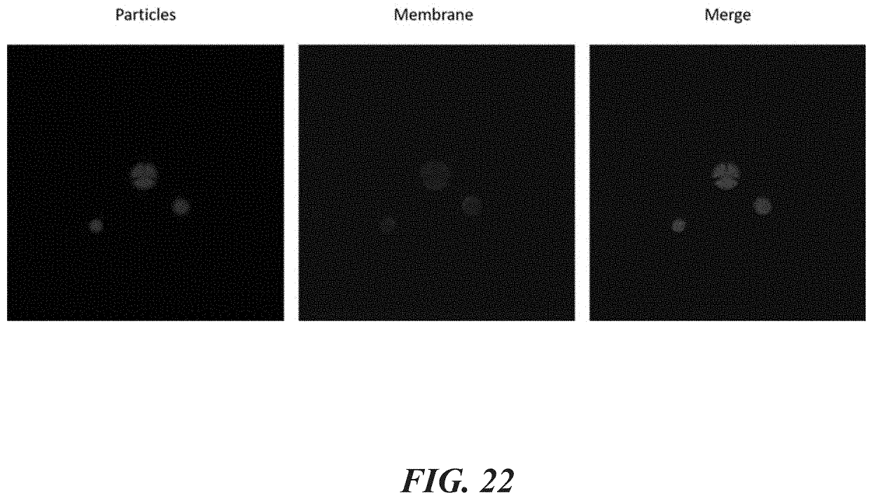

To analyze the formation of supported biomimetic membranes, confocal imaging of PLGA particles encapsulating 7-AMC were coated with rhodamine containing membrane derived vesicles. Confocal image acquisition was completed with a Zeiss 780 FCS Confocal Microscope. To derive profile information, we used the ImageJ profile measurement tool and drew a line through the particle to determine relative fluorescence information. Characterization of RBC, platelet (resting and activated), and dendritic membrane coating on PLGA particles can be seen in FIGS. 9-22.

### Surface Protein Conjugation and Characterization

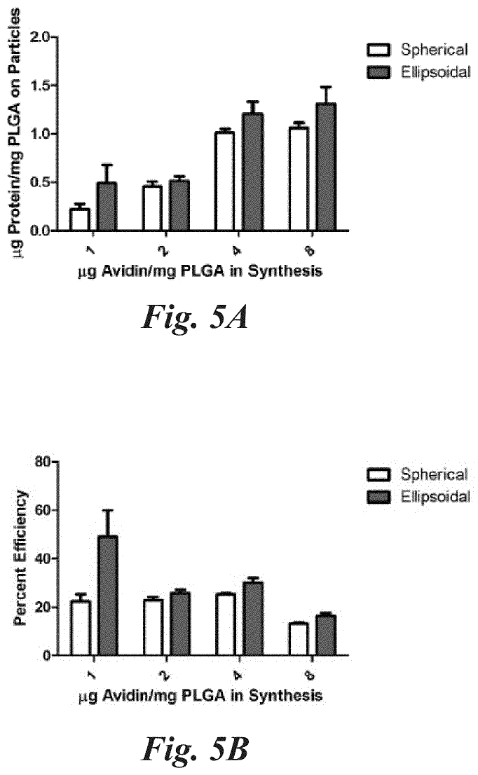

In order to functionalize the supported lipid bilayers to be receptive to any form of protein conjugation, we first functionalized the surface with thiolated avidin (Protein Mods; Madison, Wis.). We first were interested in whether or not the avidin could conjugate to the surface of the maleimide activated particle. We pre-reacted biotinylated fluorescein (Sigma-Aldrich; St. Louis, Mo.) with the avidin and then dialyzed overnight with a 10 kDa MWCO dialysis bag (Life Technologies; Grand Island, N.Y.). The particles were then reacted overnight with various amounts of fluorescent avidin and washed three times. Fluorescence intensity was measured under a plate reader and correlated to the amount of fluorescent avidin on the surface of the particle. Characteristic avidin content is shown in FIG. 5A and FIG. 5B.

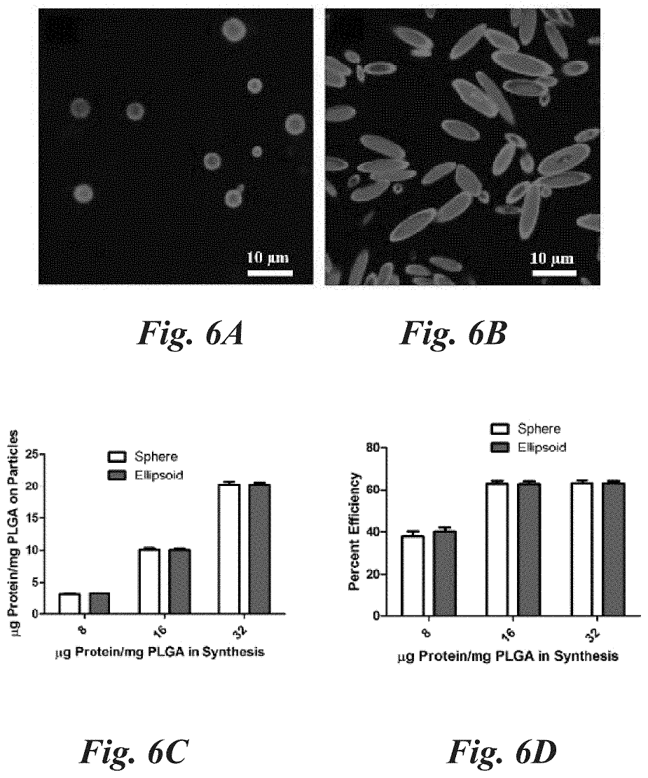

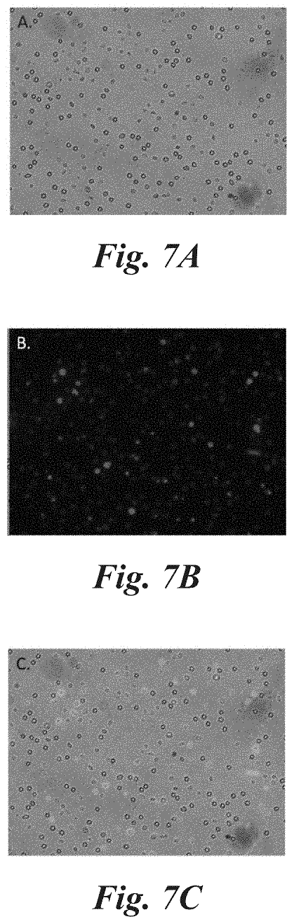

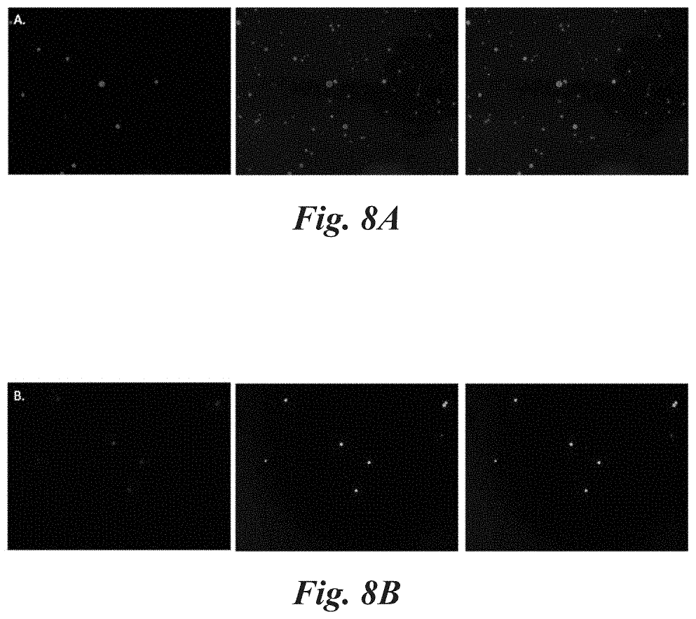

To evaluate our capabilities to conjugate a target biotinylated protein to the surface of our supported lipid bilayers, we formed the SLBs of ellipsoidal and spherical shape and conjugated them to the thiolated avidin overnight at 4° C. at a 4 μg avidin/mg PLGA ratio. We then conjugated Cy5-biotin (Click Chemistry Tools; Scottsdale, Ariz.) at a concentration of 4 μg Cy5-biotin/mg PLGA ratio for 1 hour at room temperature. After washing 3 times at 4° C., we evaluated the conjugation through confocal imaging of the particles. Representative images are shown in FIG. 6A and FIG. 6B.

To confirm that this method would work for a bioactive protein, we utilized biotinylated anti CD28 as a model protein for particle surface capture. Avidin functionalized SLBs were prepared as previously, but instead of a fluorophore, we added the protein in at various concentrations to test reaction efficiency. To quantitate the amount of protein bound to the surface, we washed the particles 3 times, collected the supernatants, and analyzed them for a reduction in protein content utilizing an Octet Red system (Forte Bio; Menlo Park, Calif.). Reduction in protein content in the supernatant was then converted to protein immobilized on the surface through subtraction from the total amount of protein added into the system. Representative protein content is given in FIG. 6C and FIG. 6D.

### Synthetic Lipid Fusion for Particle Visualization

To illustrate the capability of the red blood cell membranes to be integrated with synthesized functionalized lipids, we adapted a protocol previously developed for the insertion of fluorescently labeled PEGylated lipids in to RBC ghosts (Fang et al., 2013). Briefly, RBC ghosts derived from 500 μL of whole blood were incubated with 50 μg of PEGylated DOPC (to promote membrane fusion) and 50 μg of DSPE-rhodamine. After 30 min at 37° C., we then washed the ghosts 3 times and imaged with fluorescence microscopy to illustrate ghost integration. The ghosts were then processed into submicron vesicles as described above and then coated as described above. Fluorescence microscopy of the coated particles was used to confirm that the synthetic fluorescent lipids were integrated as part of the red blood cell membrane derived coat.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Anselmo et al. “Platelet-like Nanoparticles: Mimicking Shape, Flexibility, and Surface Biology of Platelets To Target Vasculature,” ACS Nano. 8(11) 11243-11253 2014.
Champion et al., Proc Natl Acad Sci USA 2007, 104, 11901-11904.
Fang et al., Nanoscale 2013, 5, 8884-8888.
Ho et al., Colloid and Polymer Science 1993, 271, 469-479.
Hu et al., Proceedings of the National Academy of Sciences 2011, 108, 10980-10985.
Hu et al., Nature 2015, 526, 118-121.
Little et al., Proceedings of the National Academy of Sciences of the United States of America 2004, 101, 9534-9539.
Meyer et al. “An automated multidimensional thin film stretching device for the generation of polymeric micro- and nanoparticles” JBMR Part A. 103A(8) 2747-2757 2015.
R. H. Fang, C.-M. J. Hu, B. T. Luk, W. Gao, J. A. Copp, Y. Tai, D. E. O'Connor, L. Zhang, Nano letters 2014, 14, 2181-2188.

Although the foregoing subject matter has been described in some detail by way of illustration and example for purposes of clarity of understanding, it will be understood by those skilled in the art that certain changes and modifications can be practiced within the scope of the appended claims.

1.-26. (canceled)

27. A biomimetic particle comprising:
(i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and
(ii) a naturally derived cell membrane.

28. The particle of claim 27, wherein the biomimetic particle comprising the three-dimensional microparticle or nanoparticle having an asymmetrical shape comprises a naturally derived cell membrane derived from a red blood cell.

29. The particle of claim 27, wherein the naturally derived cell membrane is a cell membrane derived from a platelet, a stem cell, a fat cell, a skin cell, an endothelial cell, a nerve cell, or an immune cell.

30. The particle of claim 27, wherein the surface of the biomimetic particle is functionalized with a targeting agent, a therapeutic agent, and/or an imaging agent.

31. The particle of claim 30, wherein the targeting agent, therapeutic agent, and/or imaging agent is selected from the group consisting of a small molecule, carbohydrate, sugar, protein, peptide, nucleic acid, antibody or antibody fragment thereof, hormone, hormone receptor, receptor ligand, and cancer cell specific ligand.

32. The particle of claim 27, wherein the biomimetic particle is biodegradable and/or biocompatible.

33. The particle of claim 27, wherein the microparticle or nanoparticle comprises a polymeric matrix.

34. The particle of claim 33, wherein the polymeric matrix is selected from the group consisting of poly(D,L-lactide-co-glycolide) (PLGA), poly (D,L-lactic acid) (PDLLA), polycaprolactone (PCL), polyglycolic acid (PGA), polylactic acid (PLA), poly(acrylic acid) (PAA), poly-3-hydroxybutyrate (P3HB), poly(hydroxybutyrate-co-hydroxyvalerate), a poly(beta-amino ester (PBAE), and blends thereof.

35. The particle of claim 33, wherein the polymeric matrix is stretched from above the polymer transition temperature up to the polymer degradation temperature to form an anisotropic polymeric matrix.

36. The particle of claim 33, wherein the polymeric matrix is stretched at a temperature above but close to the polymer transition temperature to form an anisotropic polymeric matrix.

37. The particle of claim 36, wherein stretching of the polymeric matrix causes the polymeric matrix to change from a generally spherical shape to an anisotropic shape.

38. The particle of claim 27, wherein the biomimetic particle further comprises a biodegradable polymer or blends of polymers blended with a nondegradable polymer.

39. The particle of claim 27, wherein the biomimetic particle ranges in size from about 10 nanometers to about 500 microns.

40. A kit comprising the biomimetic particle or composition of claim 27.

41. A method for delivering a therapeutic agent to a diseased cell and/or tissue of a patient, the method comprising:
(a) administering to a patient a biomimetic particle of claim 27 loaded with a therapeutic agent for treating a disease or disorder; and
(b) allowing the biomimetic particle to target a diseased cell and/or tissue of the patient,
thereby delivering the therapeutic agent to the diseased cell and/or tissue of the patient.

42. The method of claim 41, further comprising administering to the patient an effective amount of a biomimetic particle loaded with a therapeutic agent for treating a disease or disorder in the patient in need thereof.

43. The method of claim 42, wherein the disease or disorder is selected from the group consisting of excessive bleeding, thalassemia, thrombopenia, thrombasthenia, cancer, an infectious disease, and a degenerative disease.

44. The method of claim 41, wherein the therapeutic agent comprises a biotinylated drug.

45. The method of claim 44, wherein the biomimetic particle is conjugated with a biotin-binding protein or fragment thereof.

46. The method of claim 45, wherein the biotin-binding protein or fragment thereof is selected from the group consisting of avidin, streptavidin, neutravidin, and fragment thereof.

47. The method of claim 44, wherein the biotinylated drug is a biotinylated antibody.

48. The method of claim 47, wherein the biotinylated antibody is specific for CD28.

49. A method for imaging a diseased cell and/or tissue of a patient, the method comprising:
(a) administering to a patient a biomimetic particle of claim 27 further comprising
an imaging agent and
a targeting agent;
wherein upon administration, the biomimetic particle targets a diseased cell and/or tissue of the patient; and
(b) imaging the diseased cell and/or tissue of the patient.

50. The method of claim 49, wherein imaging the diseased cell and/or tissue of the patient occurs by bioluminescent imaging, fluorescent imaging, x-ray computed tomography (CT), magnetic resonance imaging (MM), positron emission tomography (PET), single-photon emission computed tomography (SPECT), and/or X-ray, and operable combinations thereof.

51. The method of claim 27, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (f) about 20 μm to about 100 μm; and (g) about 101 μm to about 1 mm.

52. The particle of claim 29, wherein the immune cell comprises a dendritic cell or a T cell.

53. The particle of claim 30, wherein the targeting agent, therapeutic agent, and/or imaging agent is selected from the group consisting of a polypeptide, an antigen, an enzyme, an amino acid, a hormone, a steroid, a vitamin, a drug, a virus, a polysaccharide, a lipid, a lipopolysaccharide, a glycoprotein, a lipoprotein, a nucleoprotein, an oligonucleotide, an immunoglobulin, an albumin, a hemoglobin, a coagulation factor, a peptide hormone, a protein hormone, a non-peptide hormone, an interleukin, an interferon, a cytokine, a peptide comprising a tumor-specific epitope, a cell, a cell-surface molecule, a cell adhesion peptide, a cell-binding peptide, a cell receptor ligand, a small organic molecule, a small organometallic molecule, an oligonucleotide, and a transferrin.

54. The particle of claim 30, wherein the targeting agent is selected from the group consisting of a sugar, a peptide, an antibody or antibody fragment, a hormone, a hormone receptor, and a receptor ligand.

55. The particle of claim 30, wherein the targeting agent comprises a moiety that has affinity for a tumor associated factor, a low-density lipoprotein sequence, a NAALADase inhibitor, an epidermal growth factor, an inflammatory factor, a cytokine, a cytokine receptor moiety, or a TNF-α receptor.

56. The particle of claim 30, wherein the targeting agent comprises an antibody targeting an antigen or an immunogen.

57. The particle of claim 30, wherein the therapeutic agent is selected from the group consisting of a small organic molecule, a small inorganic molecule, a chemotherapeutic agent, a saccharide, an oligosaccharide, a polysaccharide, a peptide, a protein, a peptide analog or derivative, a peptidomimetic, a DNA, an RNA, an siRNA, an shRNA, an antisense RNA, an miRNA, a ribozyme, a dendrimer, an aptamer, an antibody, and combinations thereof.
